SEQUENCE LISTING (2 sequences)

SEQ ID NO: 1 - DNA, 401 bp, Homo sapiens
ccccaaatttaaaagagggcaagaaaagagagacaatagaacataaaacaaatgggacaagaataaagtacatagttgtcacagtcccttgggtgctgctgtaacaaaatacctgagactgggtaatttacaaagaacagaaatttatccattcatggttctggagtctgggaagtccaagattgaagggctgcatctggwgagggtcatctggctacattataacatgatggaaagcatcacatggtgagagagagcaagagaggacagaacttacttttataacaaactcattctcacaataaactgctcccttggtgataacattaatccatttatgagggtacagccttcataacctaatcacctcttaaaaggttccacctctcagctcgcagtga

SEQ ID NO: 2 - DNA, 33164 bp, Homo sapiens
  Unsure (12165)..(12165): n is a, c, g, or t
tgactctgttattaagatttttaccaaagagcaactgtagctatggtggcctgacttgaagggacagaccaaacaaacaacaaaacacaaaacaaaacaaacaaacaacaaaagcaagagcagaaactattggaaaatgatttattcaccaatattttaaaggttcttcctgttctaagcaaggaacatctttaagttctttataatgtacccattttacatttacaaaaactgccagaagttgacagcatgttttgggggtttctaagctatcagttaattttttggggtacagttttattgataaactaacagagtagaaaaaatctaatggaatacccccaaatttaaaagagggcaagaaaagagagacaatagaacataaaacaaatgggacaagaataaagtacatagttgtcacagtcccttgggtgctgctgtaacaaaatacctgagactgggtaatttacaaagaacagaaatttatccattcatggttctggagtctgggaagtccaagattgaagggctgcatctggwgagggtcatctggctacattataacatgatggaaagcatcacatggtgagagagagcaagagaggacagaacttacttttataacaaactcattctcacaataaactgctcccttggtgataacattaatccatttatgagggtacagccttcataacctaatcacctcttaaaaggttccacctctcagctcgcagtgagccgagatcatgccactgcactccagcctgggtgacagagcgagactctgtctcaaaacaaacaaacaaacaaaaattccacctctcaacactgttgcattgaagatgaaatttccaatacatgagctttggggcacacattcaaaccatagcaatagttcaattactttaaagcccaaaatgtcagtaattatattaaatgcaatttaatacatcagtaactaaatgagatagactaaattttatgataatcagactaatattatcagactagacttcaaaacaacccaacaatcatatgctgtttacaagagacacatttatggatactaaaaggtataaagtgaacagatgcaaccatttatcaaaagaaagctgttgtggcaatattactattagacaaaatagaattaggaaaaaaatcataactgtagataaacgtttcactaagttaaaggtttcactgagaagaaataaaaattttgaatttataagtacttataacaacattaagaaactagaaaatagaaaaagaatagagccaaaattgactgaactagaaaaatagaaaattcatagaaaaatccacaatcatagtgggagattttatcatacatctctcagtaactgatagaaaaaccagacaaaaatcaataaggataaagaaaatgcgaacacaatgtagttcacataaggaacctatactacttccaacagcagctgaaatcacagagcatacttaaaatctgaccatatactggctcatagagtaaattgcaatacattttaaagaatttaaatcatacagaatatgtcttctgaccataatgcaattaaagtagaaaagaacagaaaaacttatagaaaattctcatatatttggaaattagtaaatttctaaataacccatgggtcaaaaaataatcagaatgaaaatcagaaaatatttttaatgacttatgaaaaaaattacatataaaaacttgtcagatgcacctaaactattgcttaaaggaaacttacaaccttaagtgcatatattagaaatgaagagtgacgaaaaatgaatgagcaagcaaataccttaaaaactggaaaaggaatagtaaaataaatccagagaaatcagaacaggggaaataataatataagaatagaaattaatgtgctagaaaataggggatctatcaagtcagaagttgattttttgaaaatgctaataaaaatgatgaacctctggcaagacaataaaagctggtacaaataactcatctaaggaataaaattgaggacctctcttttgttaaatgacataagcaggaggccattatcctgagactgtctctacactttgagttcttacataataaactgcaacctaatttagtatactacataaacaaactgaaacctaacttaggagtctttttttttgtaattaatagctaaatttccaccaaatcataaatagccaagctttagccaatcacaggcagccaactgatcagactatgcccaaacaaggtaaatgcctattacaccatacccaaataaggcagactcctagctgaagccaatcaggtgattactatgctcttgtgtttggcctataaaagctcactgctcacactgttggatgctctctgaaacttttgtgaatctgagtgctgcctgattcatgaatcattctttgctaaaataaactctgtcaaatttaatttgtctgaagtttctcttttaacagtgtttggtgttgcaagcaggatccaaaggagacctatagtgactcccaggagacctatagtgactcccaggagctcttagtgaccaagtgaaggtacctgtggggctcattgtgcccattgctctttcactgctttcaactggtagttgtgggtaagttcctgctcagacttgagcactatacatttgcattttcagctctctgactttatttgagcaattctttgatggattggctccagaattggattggattcagtgaagaaactgaaactggactggtgctctaggaggcctcaagtagataaggttactagaagacagggaatcctgggcttatctggatttaaggagtctacaactccatctgcaactccagccgatttcatttataagaattatggaccaagaacctgtgcttttctagagaaatgtgtgagccttactaaaggtaacttagaattacaatggactgccgagaccagctcggtcgtggagaccctaaccccagcagcgctagaggaatcaaagacacacacacagaaatgtagagtgtggagtgggaaatcaggggactcacagccttcagagctgagagcccctaacagagatttacccacatatttactgacagcaagccagtgataagcattatcgctatagattatagattaactaaaagtattccttacaggaaacaaagggatgggccgaaacaaagggataggctctggctagatgtctgcagcaggaacacgtccttaaggcacagattgctcatgctattgttgtggcttaggaacacgttaagcggttttctgccctgggtgggccaggtgttccttgccctcattccagtaaacccacaaccttcagcgtgggcgtcatggctgtcacaaacatgtcacagtgctgcagagattttgtttatggccaaatctggggctagtttatggccaggtttgggagcctgttcccacatgtcccccttttttgttttgcaaagcaataaaagcaaaggcagctttgtcacagtgagctacttcttgcaggagtcgggattcgcatatgcatactatacaaagacaaacaacacagattaaaagcccaaccatcattgaaatcacagagcttccaagtgtctttatctattttaatgggttaatcactgctaatctgtctgcagctccttcaagcactccagttcctggcattaaggtctggtgtgcctggaatgctttaaatatttgttcttttaatattgcaatatggaaagacaagtttgtagagtgtcttctaggtgcttttttattctttcctgaatttggtcttatttaagagccattaatagtttccacaaatccttatgtttagctcctacagcgggccatatcatttgagggtgaggtgccactataccatcatgtttccagataataggaactcttgccatatttcttgctatttctaccatctgaccattttgtttagaccagctaaacatagtgtggccatggcacgcagactgagaggtgcaatttaagctaaacatccccttaggggaccaatcaataatgattccataggaatcactgcgcagcacctctgcctattctgcaatgcaatcttcccaaacaagtatgttcattatttctggccaggtccaattctgtttacaaataggtttttgagggcaatatgcctcaattatagaaacagatttattatggtaaatactgagatcagaaagcatgtgtaactgtgtcatagaatgattacatccaggcattattgccagccaagattgataaatatgcccaataagtataattgttctctgtgttagcccttgttgaaggaatactaatggcaatggtgatcactgctatcatagctatcattaaattactcattgtgactggttgtcccactttcctcaggttttcttccaccctctatggcagtttcttgatctgtccccaggtagatggctgtgtttgatggttgttgctcgtgatagtttgggtcctcctcagcatcagtctcgacatggctgcaaccagagggtcctcgggatcctcccagaatctcttccttggcatctggctcatgataaggtttcaggtgtcttgatggtatccaaatcagctattgattctggcctggagaaacacaagcataacctctaccccaagttattattttacctatttcccaactttttgttgtcagatctctgcaccaagccagttgttctgcttctgtttttgcagctggtttctgtagatgctcttcagctgctgatagcatctggcctttaggcaggctcaaaaaatttaaagtcattaatgctagattcagttgtatatgggtgtcctgtaatccctgtttcccctttttgcttttgcaactgctgtttcagggagagattcattctttctacaatggtttgtccttgagaattatatgggatgccagtaatgtgtttaatattccatatagagaaaaatgtagctagagcttggctagtatagcctggggcattatctgttttaacagaagttggaattcctatcaccacaaaacactgcaaaaggtgatgcttaacacatgcagtctcctgattggcatgtagcccagacaaagtgagaaaaggtgtccacacatacatgtacataagctagtctcccaaacgagggaacgtgtgacatccatttgctaaagataattaggttccaatcctcgaggattaactcctcctgttaaagatgaggaatgcaccatttggcaagttgggcatcactggataatagctttagcttctttccaggtactgttatatctgcatttgagaccagaggcattaacatgggttaaattgtgaaagtgtctagcattagatattgcagtagcaactaggcgatcagccatttgattcccttcagttaaaggtcctggaagaggtgtatgagccctaatgtgagtgatgtaaaaagggtgcattctactcctaaatgctgtttgcaattgggtaaataaagtcatcagttgttcatctgtatgaagtcataactgagcattttcaattaattgtatggaatgaaccacataggaagaatcagaaatcacattaataggcatatcaaaagcagtcaatacctcaattacagctaaagctccactttttgagctgaagtatagggcgtgtgaaaaactttaccttttgatccagaataagaggctttaccattacaagacccatctgtaaaaacattctcagcaccttcagttggtttaaatttagttattttggggagaatccaattagttaatttcaaaaattgaaataatttcattttaggaaaatgattatcaagaatacccacaaagtcagctaaatgggtttgccaagtaagactatttatagaagcttgctgtatttgtgcctttgtgagagggacaataatttttccaggatcatatccatgtaatttaacaatccgaattctcccatttcttatcatcgtagccatttgatccaaataaggagttagagtctgtgaattagtatgtggaagaaaaagccactgtacaaagtcttgctcttatacaataacaccagtaagtgaatgctgagttggaaaaattagcaaatctagggtcttttttggatctattctatttatttgagatttatggatttgctttttgattagctgcagctctgcctcagcttcttttgttaattgctgagggctaatgagactaggatctcctctaaggatagaaaatagattactcagggcataggtgggaatgcctagagcaggtcatatccgattaatgtcccctagtaatttttgaaagtcatttaatgttttcaattgatccctacgtatagttactttctgtggcacaatggtagtgtcatttactaaggtccccaagtaggagtaaggagtagtagtctgaattttgtcaggagctatataattaaactggcatgagaaatcgagttttgcaagtgatcataacattggagtaatatttcttgagtgggggcagcacaaagtatattatccatataatgaataatgtaacactgtgaaaattttttacgagtaggttcaattgcttgcccttcataagtctggcaaatttttgaactgtttcacatgccttgtagcaacactttccaatacaaatgctttagtatgctgcaggttttttactgcaggaattgtaaatgcaaacagttcacagccttgctcagctaaggggatagtaaagaaacagtcttttaaatttatgactattaaaggccaattttttggaatcatagcagaagaaggcagtcccggctgcaatgtccccataggttgtataactgaattaatggctcttaagtcagttaacattctgcatttacctgattttttcttaattacaaaaactggagaattccaaggggaaaatgttgagctatgtgtcctttttctaattgttcagcaactaagtcctctaaagcctccagtttctctttactcagcagccattgtgctatccaaattggcttatctgttaaccattttaaaggtatataggttctggaggcttaacaatggccaccatcaaaaatgatatcctaaaccttggtgggaactttgtctttccgcttgaagcatttccttcaaagcttgcaaattttttcctagtcccataccagggacataccccatttcatgcatcatatgttgactttgagggctgtataattattctggaattagaacttgtgatccccattgttgtaataaatctcttccctataaatttataggtacagaagttataattttttgtatagtcccaggttgtccattgggcccttcacaatgcaaaatataactactttgatatactttaagggctttaccaactccaactatgttaaattgagcaggttgaattgaccatgcagatggccagtgctgtacagaaatgattgaaatgtccgctcctgtatctaccaaacttttaaatttctttccctgaatagttatttcaccagtaggatgtttatcagtaatttgattcacccaataagctgctttgccttgtttatttgtgcttccaaatcttcctgttcatttaatttcacttttccccatttccacatatggcataatcaggagctgtgctatatgctctcctggctctgctttccagggaacagaagtagatataacaatttgaatttcccctttgtaatctgaatcaatgactcctgtttgtacttgcacgccttttaaatttaaactagacctacctagaagtaatcctaccatccccgctggcatcatctgactggagacagcaaccttctttaacagtcccattacaaaaggagaacctgttccatatggattaatagcttgtttaaattctttgagtaatttaaaaggaaaaggctcaaatgtagctataatatttccctgttgatctacggggtatattctaacagggaactgctaagcctctatatcaccctctcttctaacttgctgaattcctggctgaatagaactgagagtggtcgctcgaggtgctgctcagtcactggggcgactactttttgcccagtgtcctctggaaaagaaagatctggaggatcaggctactcttttgcttcgaaataatgagggggtgcagaagggtagggacaaacctctccctcctttgccactttagctttagctggcaaacaaacctgctctgtcacctcttctgttacttcattatactttccttcctcctcatctttagtgtgaaaaggttccaaggtggaatgagctagagcccacacttgcccattgttaccctgatgcttccgagctccccttcttactcaccatggggattgcttaagagtactcgggtatcctccagctttgttccacgttttccaaccattgctccagcgagccttcatcctgggtttgagtccccacgttgggcgccacttgccgagaccagctcggtcgtggagaccctaacccagcagtgctagaggaattaaagacacacacacagaaacatagagtgtggagtgggaaatcaggggattcacagccttcagagctgagagccctgaacagagatttacccacgtatttattgacagcaagccggtgataagcattatttctgtagattatagattaactaaaagtattccttacaggaaacaaagggatgggccaaaacaaagcgatgggctctggctagatatctgcagcaggaacacgtccttaaggcacagatcgctcatgctattgtttgtggcttaggaatgccttaagtggttttccatcttgggtggaccaggtgttccttgccctcattccagtaaacccacaaccttcagtgtgggcgtcatggccatcacaaacatgtcacagtgctgcagagattttgtttatggcaagatctggggccagtttatggccagatttgagggcctgttcccaacagtggacaaagaagaaaagctgcttaaggcaaacaggcacatattttttaaagctacaatagctagacaaactaagagaatcctgtgtatgttttacttttgtatttttatttttaatatttgtggatacatagtagatgtatatatttatggggcacttgagatattttgatacaggcatacaatggataataataacatcacggtaaaaggggtatccattacctcaagcatttatcctttctttgtgttataaacaatccaattaaaccttttatcataaaatgtacaataatgttgactgtggtcaccctgttttgctatcaaagaataaatcttattcattttatctaagtatatttgtgtacccattaaccatcccagtccccctgctcctccaactacacttcccagcctctgttaaccatcattctactctcttatctccatgagttcaattgttttaatttgtagctcccacaaataagcgagaacatgggatgtttgtctttctgtgccttatttcacttaacataatatcctccagttccatccatgttgttatatgacagaatttcattctttcttatggctgaatagtaccccattgtatatatataccacattttctttatccattcatgtgtaggtggacacttggattgcttctagatcttggctactgtgaatagtgcagcactaaacatgggcgtgcagatatctccttgatatattgatttcctttattttgggtatatacctagcagtgggattgctggattgtgtggtagctctactgttagtcttttgaggaacctccaaattgttctccatagtggttgtactaatttacattcccaaccaacaacagtgtacagggttctgttttctccacatccttgcagcatttttattgcctgtcttttggataaaagccattttaactggggtgaaatgatatctcattgtagttttgattagcatttctctgatgatcattgatgttgagcacctttttatatatctgtttgccattcatgtgccttcttttgagaaatgtctatttagatcttttgcccatttaaaaaattggattattagatttttttcctattgagttatttgatctccttatatattctggttattaatcccctttcaaatggatagtttgtgaatattttctcccattctgtgtattgcctctgcactttgttgattatttaatttgctatgcagaaactttttaacttgatgtgatcccttttgctcatttttgctttggttgcctatgcttgtggggtatgacttaagaaatctttgcccagaccaatgtcatggagagttttcttaatgttttcttttaatagtttcatagtttgatgtcttagatttaagtctttaatctattttgtttttatttttgtatatggcaagatataggggtctagtttcttccgcatatggatatctacttttcccagtaccatttgttgaagagactgtcctttctgcaatgtatgttcttgacacttttattgaaaatgaattcactgtagatgtgtggatttgtttctgggttctctattctgttctgttgttctgtgtgtctgtttttatgccagtaccattctgttttagttactatagctctgtagtataattttaagtcagataatgtgattcctccagttttgttctttttgctcagaatagctctggctattctgggtcttttgtggtttcatgtaaattttaggattatattttattaacatatttctgtgaagaatgtcattggtattttgataggaactgcattaaatctatagattgctttgggtagtaaggatattttaacaatattgatttttccaatccatgaacatggaatatctttccattttttggtgtcctctttaatttcttgcatcagtgtttatagtgtttctttcatcagtgtttatagttttcattgtagagatcttttactcctttgattaagtttattcctgggtattttatttgtagctgttgtagacgagattactatcttgatttctttttcagattgttcactgttggcatatacaaatgctactgatttttgtatgttgattttgtatactacaatttcactgaatttgcttattacagagagactaaagatagttaggtccattaccaaatgtgttttctgtcacattgaaaaattataccatgagaaggcacatgcttctagtaattacagttcatagatatgtcaatgtacagaatgctggtgtgacagttcacaattgtttgctttatagttttcactggaaattaaattactaagggtaaagtaatcccagcactgtgggaagctgaggcaggatgattgcttaatgccaggagtttgagaccagctggggcaatgtagcatgagcccatctacaaaaaaaaaaaggaattctaattaatatgtggtactagaattaccagaaataataagggaaacaactcggaaaagaaggtaagatgtgttttgggtaagaacagctataaagtatgaggggatgttttgttttgttaaggaaaaaataaaataatatttgtcctaaagtagaatgcctggttgttaagacaatgagaaagagaaagagtatagaagaaaaactgaatggatataagaaagttgtagaaggttgtgggataggaatcctggaaagggaattttatatgtgatcaaactaagatttgaagaaaattctaagtttttttttaaaaaaattgagcatgaatatcaaaaacatactgacacagaactagaaatttgtcctctatgttaaaacaacaaggttttcttggagtattggtctgctcttaataagaaattgaaagacttttctttacctgctaagtaattggcctaggaaatgaagattctgtgttttattgagataatttattgtgcttcatgttgtctttattagatctttgactgcctcagagacctgagttcaccttattaaaagagaaagagtttctacaattatgtggctttctttatttgcctttgaagtcttttaccaatcactctagttaaatgaataaatattgtttcatagggacctgtgatcctattttaatcagatattttaaaccttntgatatttttgacttctcaaaatcaaattctaaattaaaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggccttcatctataagtttttccttacccctttggtgggccccctcagcccagctttttttttttttaaaggacagggtctcactctgttgcctaggctggagtgcagcagcacaattttggctcactgcagactcaacctccagggctcgagtaatcctcccacctcagcctcccaagtagctgggatcacaagaatgtgccaccacacccagctaattccttatttcttatagagacagagtcttactatattgcctaggctggtctcaaattcctgggctcaagtgatcttcccacctcagcctctgagtagctaggactacaggcatgcaccaccatactcggctaatttttgtattcttactgtagagacatggggtctcactttgttgcctaggttggtcttgaactcctgggctcgagtgattctcctgccttggcctcccaaagtgctgggattacaggcttgagctatgtgcccaacctcctttgattttttgtttttcttctgtttgctttactcataggaaaaaacatatacatttataaatcctgattcaaagactacttttgtgtgagggtattgagtttgcttttttttctcctttcatctaggtcatgtggcctttgggagatatgtgtgtgtgtgaggggtaggatgtgtgtttaattttttaaatttatattttggtgctgtatgttgtgaaagatttgctcatagtggatcaatgaaccatcttttctaggcagttttgttgaaaatgaaggtttctctttgatttcaagaatgaccaaattaaaaaacaaaaacaaggtattaattttctattgctaccataaaaaattctcacaaacttagtggcttaaaacaacacaaacttagttttgtatatcagaaatctgacacctatctccctgggctaaaatcaagctgtggcagggctgcattcctttctggaggctctagggggcagtctgtttctgtcctggccttttctagctcctagaggccatccacattccttggcttgtggcctcttcctctgttgtcagagccaggaacgcagcatctccctgacctttcttccatctcacatccttttctctgaccacagctgggaaaggttcacttattttaaggactcctgtgattagactgggcccatcttaaggttcttaatttaatcacatattttattatgtagagtcacatattcaggggttccaggtattaggatgtggacccctttggggcaggaattataattctgcctattacaaatgatgtaagagcatatacagtacagtgtttaaaatagtaatgagttggaaataaatgttccaaaacaggaaaatacttaagttatagaacaaacaaatgttggagtactaggaagcctctaaaaatcattgtaagagaataacataggcgatgaaagaagtagaatacaaagtttataaacagtgtgatcctgtactgcatgtggggtggggtgatgccccactgggtgtgtagaattttgagtgattttcattttctttttttttttatctttcttttctggggagggggggtactgtctgctgtaagcatgtgttactactatcacatccatctcccccaaaacttttaattaaatcttaagggaaagtgtattttatttaatagtggtaaaaagtaagtatcttaaaagcaaatgctaaagtcctgataaaatgtacattatactataaaaggacataatctaaaggcaaaggcaaagggaaactttcagtaagcattccctaatagaagacctataaaatgaggtatgtagtaattacttgggctgggattaaagggacataagaatgtctttacatacaagtatagccaatatacaaaatgcacaacagtctagttaccattaatcaaaatggtgggcaagtactataaaagtcaccattaaaatacagccttattaaaatacatatatttatggaaacgaaaactgacatttcaaaataattatttctgtgtgccctagacatttgagcaatttaatacttctgctggagtacagagaggaagtcagtaagatggaatgtatttggatttcaagcagttatttttaaaggaaagctattactttagaaactaaaagcaaaacagaaaacaaaaactttaaccagtatcctttcatctagctaattagatgtttgttcattcactatagatatagaatttatttaaaacctcaaaatgattgaagtaacagactcagcactgtgactttgggattgagtaggctataagagtggggtgaaatatgaccattcattctataattcaatattaaaacaaattcctgcaaccaaagaaatagtagtactgggttaggccaggttcccaagttcattgtgtattctagatctatagtgctaataaagagaagacaataacttgtttgcctaccttatttcattgttaaaaaaaatcactaggtcaaaataatgggtcaagttgttgattgaaaaacccagaattgcttttgtatttgctgggataagctagatgtcttagttcattttgtgctgctataacagaataccacagactgactaacttctaaaaaacagaaattttcttgcagttctggagactgagaagtccaagatcaaggcactctcatctggtgagggccttcttgctgtctcatcacatggcagaaggcagaagggcaagagcacaagagagagaacccattccagagagtgcttttcataatggcattaatctattcttaagggcggagccctcatggcctaaacacctcccattagacctcacttcccagtgctgttgcatttgggattcagtctctaacacatgaatatctgggagacacattcaaactatggcactaggttatgctgtagtaacaacttattcccttaacaggcacagatgatggtccagggcaactgtcccctggtgtgaatcagcaattcaggtcacttgactcttgtgactgtggcatcacagggagacaagaggtctccctgttttcaactacagcagaaagcacgaggagtatgcagcccacctattctatgcttggcccagtggtgatacacagtacctctcttctgcttacagcacattggcctgaaatggtcgtgtggtcccactcaagggggaaggaggctgggaaatggaagcttctatgtgtcctgaaaagaaattataggcatactgctgagtgctacttatgtttaaacacacctatacaattttggagaagctttcctttattttttatccagttggaaagactctgttctctaaacttcagagctgcaggtaggacctggtcctcattctagagaaagtagctctgcagtcccagcagaaagaagcctgacttgaagctccaccactttggctatctgttgggtgttcttcaaaagtgagtgccaattcactgtagtacccggcacttggtcttccatgctttcattattagggaaactagatttgtagcacagctactggagagtactaggatcctagtaagctggctttacaaacatggtgtgtactgcctatttttgcttttatttcagagaaaataaaacgtaagataataggaacaaatacatgtgaactgctggacggctattcaaaaatagtttgccagagtaactggtaagcctgaaatctgtgtagccatgaaagggctcatcatcggcttaattctgaagcaaaacttggtgaaacacgtggtggttatcttggaggtaaaatgctggcttcccagtgggaagtgaaagtgtcaatcaggttgtgttgggagatgtgataaaggctgaggcagtcttgcagctcatgattttatgcaggggctaagatgagaaggatgttcaaaaaaaccaggggatgttattctagcttttgagcatgagacagcctcaaagcactgggaaagctggatgctagcaggaggcagtagaaatgcaagtgtgagaaaaataaaggctgggctggcgtgctggcaggaaatggattcgaaaggcaggtctgataaatgtttcagaaaaagagagctgtcaggatgaggtagagtgagcttggcagcctgggagttggcatgccattgatggtcaagaccaaatgctgtttctcctatccagccagaaattttgctttaaaaatgacactagggctttgctctcaacaaccactgtcctctctctcattctccctctcactgggttacctcctccttgtcagtgacttcttctgtgatactgactcttccatccatcatgggttttttctctgatttttaagaataagtggaaattcctatattgtttgttctttcaattgtagaaaatattgaaaaccaaaaggaaataatttaaaaacactgtaattgcactacgaagaagcacttagtttggtatacagccatgtgttgcttaatggtggggatatattttgagaaatgttttattaggtgacttcatcactgtacagatatcatagagtacttacacaaacccagattgtatagctgactacacacctgtgatatatggtatagctgagtgctcctagtctgcaaacctgtatagcatgttactgtactgaataaggcaggaaattgtaacacaatgggaatcatttgtttatttaaatatatcaaaacataaaaaggtacagtaaaaatgcaatataaaagataaaaaatggtaggtacccgtgtagggcacctaccatgaatagagcttgcaggactggaagttgctctgggtgagtcagtgagtggtaaatggatgtgaaggcctaggacacggctgcacactactgtacaattcgtaagcactacacttaggcacactaaatttataaaaaatatttttctttaataggaaattaaacttagcttactgtaatttgttactttatgaactttttaacttttaacaacttttagattgtttttaagcaacatttagcttaaaacacaaactcacgtacagctggacaaaaatattttctttttaaatatccttactctataagcttttttcattaaaaaattatttttacttttaatttttttttgttaaaaacaaagacacaaatatacacattagcctgggtctacacagggtcaggatcatcaatatcactgtcttccacctccacatcttatcctgctcgaaggtgttcaggggcaataacacgcacggagctgtcatctcctatgataacagtgccctcttctggagtacctcctcaaggaactgcctgaggctattttagagttaacttaaaaaaaataagtaggagtacactctaacaataaaaagtatgtaaatacataaacccagtaatagtctattatgattaagtgttatgtactatacataaatatatatgctatactattgtaagactggcagtgcagtaggattgtttacgtacagcgtcaccacaaacatgagcaatgcattgtgctacaacgttctgatggctatgacatcattaggtgataggaatttttcagttttgttatgatcttatggaacaactgttgtatctgtggtccattgttggccaaagtgtcatcaggagttgcatgactgtatctccttccagacttgtgtttatgtgtgtgcttttacaaaaatggaatatatacactattctaggaactccttttctcatttaatattctgtgaaaagctagacttatttatgaatgcttatgagatgttttactggcatttcacactcaatgtgacaacaccatccttatcatttctaacctatacctcatttttttcatttttcatttcattaactttttcaacatcttttgagcccacagtaggggtctaaccacttgataattgtcttagtttattttctgttgctataacagaataccatggactgggtagtttataaagaaaagaagtttatttagctcatggttctggaggctaggaagtccaagagcatggcaccaccggcatctagtgtgggtcgtcccatggcagaagcggggaaggacagaaggtggaagcaagtgcacaagacagagagaaaattgggtaaaattcatcctttttataaggcgcccactcctgtgataactaacccactttatgataatgacattaattcattaatccatataatcacctcttaaagttcccacctctcaacactgttacaatggggattaagttttcaacacaaatttttgggaggacacattcaaaccatagcaatggttaaaattaagctcagatactctcttttttccctacccttctcatatttaattgtctcaaaatcactcattcattcaataaatatttttttcatctgtccttacatggcctgtgtgcagcctgtgccctcatgattttctcattcagtgaaattgagcacctactatgtgccagactttgtttttggctctgggcataccaaagtgaaccaaatggggaaggtaccttctatcatggtgctagtattctagagggggaataaataatacatcaatatattagctatccattattgcgtagcaaattaccccaaaacttagtgacttacaataatgacatttactattttactgtgtttgtgggttaggagttggggcatgctttagcaggtcctctgcttcagggtctctcacaggccgcactcatctcaaggctcaactaggggagaattggctttcaaagtcattcatgtggttgttggcaggattccatttcttgctgtgttggctacaggctactgccaatcctggccacatgcacctctctgttagggcagctcacaatagggcagcttgcttcatcagagtaagcaagcaagcaagagccagagagagagagagaacgagagggagagtgccggtaaaacaagagtcatggtcctttatgaccaatcttgaagtgacatcccatcactataccatatcctatttgttaggaacaagtccctaggtggcccattgaaggggagggtgttacacagggtttgaataccaggaaatggggatcattgtgagccgttttagaaggctgcctcccacagtcaagaaacaagacaattccagatcatactcagtgctagagagaaaataggacagggataagacgaagagcatctgggagtgttggggaggtagtcttcattgagtgtttagagaggacctcactgaggagataacagtgaagcagagaactaaagggcaagaaagggcccacccggaaagattcaagccttccagacagaggaaacagaaaaggcaaagtggcagaatgagctctgcctgtttgaggaagagggtggagcccagtgacatggagtgaatgagcacgaaggaagtgggcgcctgctgagatgaggcccgggtaatgagtacatgaagtgaatagtgtctgtgttttgaaatactgagtaacttccctatgtcacagcttgtaagtgacacagcacacacaaggatttgaacccgagtccatctgatgctaaaatccatgtttttgccacactagtgccttgcagaatttactagaaggggatgctgctgggtggtggtcagccactccaggtgctcatgatgtaagtaagactttcagggctaaaaccaggatagtcctgaggaaattgggatagctggtcacccacaagacacgacaggaacactgagttctctgatgtgatatgaaacccaggatgagccggcagccctgagtcagcctgatgagaagtcggctgtgccttttgttaccaagaaaacaaaactgtagcataaccccaccttgttgttaaaacaaaatgtttacattcttctgttctgcttcgctcaagtcattcctgtgggacaagccgacctaacatacaggtgtagtcaggggctaggagagacctaggcaggcccttgtccattctcagtcactgaagtgagacctagggactcaggaaggactcagagttcccttagtcatcattccctctgggcatcctgcttttggcataattactgatacgagcacctcgtgtcccctcctgaattatacctacccccccgcctccccgccccctgcaattgcttagcacagtatctggcccatggaagtgacttcatggaaattcagcggatgaatgactggacaatgaacaacgcacagcaatgtgtggtgtgacagaaaaggctgcaccagcaaaaattggaagctgatctttgggtatttccttgccttggtcactttgcaatttcctgggtgcttgaaacaggaagtcagtgactctaagcccctgtgggatctggcacctcacatcaaggtatctggaaagtccttattcttcacttctccatgcgatcctaccctacagaccagccctccttttggaatctctcctgctacacattctgtgaccctgggactctctcttattttatttatttatttttatatgcccgtgtttccaaaggaataatagggcagttaaatatagctatgactgtgcctttccagagcccagaaaagtctggaaaactgggaatatccaaagctcaggatgatattgcgcacacaaaaagtactaggcagagtagtgtttagatagaaacagagtaatgtttagatagaaagcagactcagtcatataaaatatatgcatatacagcatgatctgcatttctgtgatgcttctgaagtacagaagcattcagcacttcagtaactcttattgcaagttacctcttcaatcatttttcttcaatcattaaaatgctttaaaaaattttttagcttattgaggtatgactgacaaaaattttaatatatttaaagtgtacaacttgttttgatatatgtatacattgtgaaatgattaccacaatcaagctaataaacatgtccattatctcacgttacattgtgtgagaacacttaagatctactcttagcaaataccaagtatttcgagtatatcaagtatgcaatatattattactatacactcaccatgctgtacattagagcttcagaacttatttatcttataactgaaagtttgtaccctttgaccaatgtccccatttccaccattcctagtgaccactgttctactctgtttttatgagtctggctttttttagattccacatgtaagtgagattatacagtatttgtctttctgtgactggcttattttacttagcataatgtcctgctggttcatccatgttgttgcaaatggcaagatttccttatttttttaaggctgaatactatttcattgtgtagtatacaccacatttctttatccattcattcattgatggacacttacgttgattccaaattttggctattgtgaataatgctgcaataaacataggagtgtagacagctctttgacatattgatttcattttccctgggtatatgcctagaagagggattgctggatcatatggtggttctggttttaatattttgaggaaactccatattgttttctatgatggctataccaatttacattcccgccaataatgtataacagttccctttctccacatccttgccaacacttatcttttggctttttggtactagccatcctaacaggtatgaggtgatgttttattgtggttttgatttgcatttccctgatgattagtgatgtagagcacatttttatatgcctgttggccatttttatgtcttctttgggaaaatatctattcagattattttcccactttaaatccagttatttgtttttttgctattgaattgaattttttatgtattttggatagtaaccctttattatatataaggtttacaaattttctcccattctataggttctttttttctttattttactataaagtttaaaatcttgttttaatttttgtgggtacatagtaggtgtatatatttatgaggtgtgtgagatgttttgatacaggcaaattgtgtcacagaaaatcccctcaagcatttatcttttgtcttatgatacaattaaactcttagttattttaaaatgtacaattaaattatcgactatagtcaccctgctgtactatcaaatagtcggtcttattcattctttctaactattttttgggtacccattaaccatacccatccccccgcctcccccttacccacctctataggttcttttcattttgttaatttttttcctttgctgtgcagaataaaatgctttttcaaagcaagttaaagctgctctctacttagattaagaaatcaacttaaagtattatatatatatatatatatatatatattaaaaaaaaaaagaaaacatctgaagtggctccagcagaaggaaaggcaagatgggaatcaaagtttaatctacattctttctgcttctccttttctaccaacactgtgttcaactgcataacaatagtaccaatgagcacgagaaagatagtttttagtttaaagactatttcagttttagtcagactacttcagacctcagactccagacttcagactccagactttagacaacagactccacatgccacacaacagactttcagactccagcttcagactccagactccagatcgcacacttcagacaacagactctagacaacacactccagtctccacattccacacgacagactacagactccacactccagacaacagactccacactccagacaagagactccacaatccagacaagagactccacactccagacaacaggagacttttagactccagatcagacttcgaacttcagattcactccagacttcagactccagaccagacttcagactccagacccagatttcagattttagacttcagactccagaccagatttcatatttcagactccagaccagacttctgacttcagacttcagactcctgaccctggatttcagactccaggcttcagagtttggactttggactacttcagctttagtcagacagacaacttcagtcacaccacttttcacctctgccatctaatctcgtcttttgaacaaccccctaaaaaggtaacattcccaagaaacccagacccagaaacactgaatgacatactcaagactacccaactagtaagggggaggccacagtttcccttaccctggcaacatgcacatggcaggcccttcacttagctggtagaaggaacacaggcttgggaaggagatgtctgatgttcactccctgacgacactcttacttgcttcaatgatctctctgagccttgggtggcccatctgctaaacaaggatgagttatttcctggggatgctaagagacttctgcctcatgcctgcagtgctgctcatgttgcccccttggaattacttgttcaacttctttcttccccactagactgtgagcttcttcaggtcaaagaccaggactttcttgccattaaccccagcaactagcatcatgcctgaccaaagggcatgctgatgacaataaacattgattgtccgaaatcacatgcctggtccccagtaggcgcttgctggttactgctttcttcccatcctccctctgccaccccttcctctctgagtctggaggggatggccatgtactagccaggggcagtgtccacaggaaattcaggtcctttgcagcagtcagtttcatttcctatgattaaaatttctgtgaaacatattatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggtatgttattaatatgtgcttcaaaaattatatgaaattcttagaaaccagatagtcgtggtataatactatcagccaaaattctaattatcgtgaaatattatgtgttgcagaagtaatacaatttccttatgaatggcattattgtgaactctcatcatatctttaaccaagtccattttaagctttgtcatctacaaagagtcacttgttttactctggtactttccggaacactgtgaaaaacaattatataaagggtgtggacctataagacaaacaaccaaacaaaaatcgtaaagtgttcttttaaaagattcgtggaaaggactctgataagtacaggtttctgataactttaagatcatactactggactgggtaagaatttccagaactctagcaaagaagtggattggttcataaaactgccaacccaacatgaagcagaataagaattaattgaataccagggaaatgctttggcagattttcatgctaagtcagccagtactgaaattgttaagatatgcaatttgaaagaacactataatattcatctgaatcaaattatctatgataacctatttaaatgaacagcatcatgcacctgaattggagaaacaaaactgatatttaaaatgatataaatttagtgttaaatgtggcctcatggatcagacagctatctggtcctttctgagtccatatagctgccattattaaaagttctgcactctatgactcatcacagaatagataacgtgaaattatgaaaaaatattggtgttatgactgttctaaaattgctaaaatggcttataatcaatgtttggtttgtcaaatccacaatttttataagataataaaaacctcagttggtacatttccaccatccactggactatttgaacatttacagatggattgcattcagttgcctcattgggaatctgcaggaaacttaagtaaatgctggatatgtcatgttgaaccaaatactaatatgcttttaaagatcttctaattcaccctatagcagatttcactgatattccaagtgtgactacctgttcaaattgcacatctggtctttctatagggttaggctttttgagtcacatattcagatttcatgtttaaagctaacagtagacaaaacatataggagaggctttacagttaaattattccagaaactgaataataaaaccaaaaggagagataattagacagtttgtagacgaattcataaccctaattctctaattgttggtagcctccattgatgataattaatagtggagccttagataaatactactattgcttccccgttgatgacccctgtagcacacagtgtcccacagggaactgtctcttgtgcccctctagcatatatttttatctatgaaggatttaactatcaatcataggcacaggcaactccatgtcccagtcagtaggaaataagaagtcagtgtggattagggattctaacagtaccactgtcactccataaccaactgaaaatttgacattgttctatacctcttaatttgcactatggtataaaaaggaactttctagcaggtacgaatccctctaaatgggcatcttttgttcaaatgctccttccctggcttggaataaatgtaaatgaggttatgattagaaatccccttcaaacattagctactacagctcactctactgcaaaggctatatagttgcccagtgaagttctctaaattctcttgctaaagtttctttaaataacaggattgccttgaactatctgttggtttgtagtggggcagggggtgtgataggggacactgtgtataatagctaatacatcctactgcacttgcataaaggaatctgttataaagaaaatgaaataaaggtataaaagttggcaagaagaaaaccatcattatgtcttgaggatgattggggatacagaaaatgagaagaatccacaaactattagaattactaagttaatttagcaaggttgctcaatgcatggtcagtatacaaaatttaattgaatttctacatatatgtatatgccatattgtgttagattgttttgcattgctataagggaatacctgaagctgggtaatttataaagaaaagaggcttatttggctcatggttctgcaggctgtacaagcatggcactagcatctgtttgaccttaggaagcctttactcatggtggaagaagggggagcaggcatgtcatatagtgagagagggagtgagagggagggagcaagagtgaagggggaggtcccagactctttttaaataacctgaaatcctgtgaacagagtgaaaactaacttattaccatgaagagcacaccaagccattcatgaggggtataccttcatgacccaaacgctttccaccaggtcctaccttcaacactggggatcacatttcaacatgagatttggagaggacaaacatccaaatcatatcacatatataaatatgtaaaactattttaaatgtttttaaagtatcgaatagaaataaatctcacaaaatatgcttaagaactttaagcaaaaaccataaaatactattgtgataagttaaagaagacctaaataaatggagagatctgtcatgctcatggattggaagattcaatactgtaatgatatgaatagttcccaaattgttctatagattcaaaacattgccagttaaaatctcagtggactttgtttttttgaaactgacaagctgattctgccatttataataatattcaaaggggcaagaataaccaagacacatttgaagaagaacaaggaggaagaagaacgtatttgaagaagaataaggaactctattaagtgtatatctcctatatattgagatacacatttgaaatacatctatcacctagatattgagatatattatacatttaatatagtgtgttgctggtatagggatagacaaatgaacattggatcagaatagcactgaaacaagatctacccctatatgggcatgtccctattaacaaaaaaatattgttgattatttcctcaagatgatctgtaaaaaacaagttaattgaacatccatatgggaaaaaagtgaatccttctgctacctcaaatatcaaccccaggtagattatagatctaaaagtgaaaggcaaagcaataaagattttagaaaataacaaagagaatatctttatgaaaatacaggaaatattttaaaaataggctagaaaactcactattaaaatatataaaatataagaacatctgttcatcaaaagtactatagtttgaatagcagtgtctcctccaaaattcatattgaaacttaatccccattgtaacagtactaatcctaaaactaagtattaagaggtgtgacctgtgggaagtgactaagtcatgaggggtctttcctcaggtatgggattagcatccttataaaaggccttgagatggaatgtagtgcccccttgccctcttgcgttctcccatgtgaggacacagcaacaaggcaccatcttggaaggagagagaagccctcaccagacaacagtgcacatgccttgatcttagacttcccagcctccagagctataataaatacatttctgttctttataaattacttggtctctggtattttgttacagaagcactaatggactaagacaaaaagattccattatgaaagtgaaaaggcaaccgcagagtggcagaagcttttggagatgatgaatatgtttacttcctggattgcggtgatgataccatgagtgtatacatatgtgtgtatacataatgtatacaaattgtgttcattgattatgtacagtttctttgtataccaattataccttaataaagctaaggaaaaaaaaaagaaaatccagaataaatatgcatggtatgacccatttatttaaaaaaaaaaagagggaatgcgaagcagtggttttggtgcgaacacaaaaattctggcttcaacagcatttaaattcccaaaccaagcatgtttgtgttttaattctttgtggccataaattgtacagctcaggcctttatagtctctcagattctgtgaatgtggggaattagttttactcataaaaagttttgttcttggggataaattttttaaaaaaatttttgtatagttagcacactgagaaaatacagacaaaggcatacagatgcaagaaatgaggtagctgaatagaccaaagaatgatatagggcccagaaggtaggcaaagagaaagttgttgggtttatggttacaagtaaactagcagttgtggtgcagatagttttatttcccccacattaatctgaacagcccatccagacttaaacactgctttttgcatttacttctaggcaggaagacagggttctgatggtgtgagtctccttcaactcagcaaaccaccttggtctgcctcgagtttccaacacccctcctgcctgctagtgataggtgtgaggcaggttgatgaacatggaacttttttcttttggtcccaaagcatgctactcctggagtttcattcagtgaatgagaactatagtttggttctgtgagatctctatgaatcaaggcggccactgaagcggagaaaagaaatgcttaaatgttaagaaagtttgaagtgcagaaaaaggtgattgtaaatccatatggttaagcttagcccatttcttaaaaggcttgattgctcattcctccattcattgatttactcactcttccaatccatgttattgagtcttgctctgtaattccggatggttgtgcttttaagtactgcatagtggttgtatgtctgtgttagcattgctgaatgtatcagggaattcatttttttatccccacacactcattcgttccacacatatgtgtgtgtctctctctctctctctctctctgtgtgtgtgtgtgtgtgtgtgtgtgtgtgtgtgtgtgtgtgtgttatgcctagaaaacatttctcaagaattagaattacgatatgctgtcaaacacaatgacttatttgaacctcttttatttgtaggttgaagcactggacaatgccacatactttgtggatggtgtgggtcttgggggtcatcatcagcctctccaaggaagaatcctccaatcaggcttctctgtcttgtgaccgcaatggtatctgcaagggcagctcaggatctttaaactccattccctcagggctcacagaagctgtaaaaagccttgacctgtccaacaacaggatcacctacattagcaacagtgacctacagaggtgtgtgaacctccaggctctggtgctgacatccaatggaattaacacaatagaggaagattctttttcttccctgggcagtcttgaacatttagacttatcctataattacttatctaatttatcgtcttcctggttcaagcccctttcttctttaacattcttaaacttactgggaaatccttacaaaaccctaggggaaacatctcttttttctcatctcacaaaattgcaaatcctgagagtgggaaatatggacaccttcactaagattcaaagaaaagattttgctggacttaccttccttgaggaacttgagattgatgcttcagatctacagagctatgagccaaaaagtttgaagtcaattcagaatgtaagtcatctgatccttcatatgaagcagcatattttactgctggagatttttgtagatgttacaagttccgtggaatgtttggaactgcgagatactgatttggacactttccatttttcagaactatccactggtgaaacaaattcattgattaaaaagtttacatttagaaatgtgaaaatcaccgatgaaagtttgtttcaggttatgaaacttttgaatcagatttctggattgttagaattagagtttgatgactgtacccttaatggagttggtaattttagagcatctgataatgacagagttatagatccaggtaaagtggaaacgttaacaatccggaggctgcatattccaaggttttacttattttatgatctgagcactttatattcacttacagaaagagttaaaagaatcacagtagaaaacagtaaagtttttctggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcactctgaaaaacttgactaacattgatatcagtaagaatagttttcattctatgcctgaaacttgtcagtggccagaaaagatgaaatatttgaacttatccagcacacgaatacacagtgtaacaggctgcattcccaagacactggaaattttagatgttagcaacaacaatctcaatttattttctttgaatttgccgcaactcaaagaactttatatttccagaaataagttgatgactctaccagatgcctccctcttacccatgttactagtattgaaaatcagtaggaatgcaataactacgttttctaaggagcaacttgactcatttcacacactgaagactttggaagctggtggcaataacttcatttgctcctgtgaattcctctccttcactcaggagcagcaagcactggccaaagtcttgattgattggccagcaaattacctgtgtgactctccatcccatgtgcgtggccagcaggttcaggatgtccgcctctcggtgtcggaatgtcacaggacagcactggtgtctggcatgtgctgtgctctgttcctgctgatcctgctcacgggggtcctgtgccaccgtttccatggcctgtggtatatgaaaatgatgtgggcctggctccaggccaaaaggaagcccaggaaagctcccagcaggaacatctgctatgatgcatttgtttcttacagtgagcgggatgcctactgggtggagaaccttatggtccaggagctggagaacttcaatccccccttcaagttgtgtcttcataagcgggacttcattcctggcaagtggatcattgacaatatcattgactccattgaaaagagccacaaaactgtctttgtgctttctgaaaactttgtgaagagtgagtggtgcaagtatgaactggacttctcccatttccgtctttttgatgagaacaatgatgctgccattctcattcttctggagcccattgagaaaaaagccattccccagcgcttctgcaagctgcggaagataatgaacaccaagacctacctggagtggcccatggacgaggctcagcgggaaggattttgggtaaatctgagagctgcgataaagtcctaggttcccatatttaagaccagtctttgtctagttgggatctttatgtcactagttatagttaagttcattcagacataattatataaaaactacgtggatgtaccgtcatttgaggacttgcttactaaaactacaaaacttcaaattttgtctggggtgctgttttataaacatatgccagatttaaaaattggtttttggtttttcttttttctatgagataaccatgatcataagtctattactgatatctgaatatagtcccttggtatccaagggaattggttgcaggatcctcgtggatatcaaaattcatagatgatcaagtcccttataagagtggcatagtatttgcatataacctgtgtacattctcctgtatactttaaatcatctctagattacttatgatacccaatacaatgtaaatactatgtaaatagttgtactgtctttttatttatattattattgttattttttattttcaaaatttttaaaacatacttttgatccacagttggttgacttcatggatgcagaacccatggatatagagggccaactgtaatctgtagcaactggcttagttcattaggaaacagcacaaatgaacttaagattctcaatgactgtgtcattctttcttcctgctaagagactcctctgtggccacaaaaggcattctctgtcctacctagctgtcacttctctgtgcagctgatctcaagagcaacaaggcaaagtatttggggcactccccaaaacttgttgctattcctagaaaaaagtgctgtgtatttcctattaaactttacaggatgagaaatactagagggtgtattttcatgtgatctggatctgtctttctggctattagataggtttctccagccatagttacttgagagagtgagtacacagtgcaaggtacagtaaattatgtatttctgtaatttaacaaaataaatactcagtttaggagaatttgaaagacacttcccacatccggactctggtcttcctcagcctctaactaccttttgagttctagttattgtccaatcactctgactctcagttcctgttttttagcctatatctcctgcatcttgttgctttttccttcttcgaaaagtcttgtttttaattctaaaaaagcctgccctttttccccttctgaatattttaaaaagtacatgtgagctaaatagatttttaagcaaatatatacctagagtttcctcatcacacaaccacacagcaacccccgcactccccctaccctaaaccaacaaaaccttacaacacacacagatcacctctacaacaacgactcatcaacacctcttttctccataccacacattcatcacaaaatataacaacaaaacacatcttattactacctactctcttcccctcaaagtatatacttaaaaaccctcctactcattatatacgatcacaaagataacgacacacatcccataatcctacctaaccagtaaccacacatcg

(19) United States
(12) Patent Application Publication
(10) Pub. No.: US 2009/0176206 A1
(43) Pub. Date: Jul. 9, 2009

(54) Toll-like receptor 2 (tlr-2) haplotypes predict outcome of patients

(76) Inventors: James Russell, Vancouver (CA); Keith R. Walley, North Vancouver (CA)

Correspondence Address: BROWDY AND NEIMARK, P.L.L.C.;624 NINTH STREET, NW, SUITE 300, WASHINGTON, DC, 20001-5303, US

(21) Appl. No.: 10/591,224

(22) PCT Filed: Mar. 4, 2005
(86) PCT No.: PCT/CA2005/000357

§ 371 Date: Jun. 4, 2007

Related U.S. Application Data
(60) Provisional application No. 60/549,560, filed on Mar. 4, 2004.

(51) Int. Cl. C12Q 1/68; C40B 40/06

(52) U.S. Cl. 435/6; 506/16

(57) ABSTRACT

The invention provides methods and kits for obtaining a prognosis for a subject having or at risk of developing an inflammatory condition and or a gram positive infection. The method generally includes determining a toll-like receptor 2 (TLR-2) risk genotype of a subject for one or more SNPs, comparing the determined genotype with known genotypes for the polymorphism that correspond with the ability of the subject to recover from the inflammatory condition and identifying subjects based on their prognosis.

### FIELD OF THE INVENTION

The field of the invention relates to the assessment of subjects with or at risk of developing an inflammatory condition or gram positive infection.

### BACKGROUND OF THE INVENTION

Genotype has been shown to play a role in the prediction of subject outcome in inflammatory and infectious diseases (MCGUIRE W. et al. Nature (1994) 371:508-10; NADEL S. et al. Journal of Infectious Diseases (1996) 174:878-80; MIRA J P. et al. JAMA (1999) 282:561-8; MAJETSCHAK M. et al. Ann Surg (1999) 230:207-14; STUBER F. et al. Crit Care Med (1996) 24:381-4; STUBER F. et al. Journal of Inflammation (1996) 46:42-50; and WEITKAMP J H. et al. Infection (2000) 28:92-6). Furthermore, septic and non-septic stimuli such as bacterial endotoxin and cardiopulmonary bypass (CPB), respectively, activate the coagulation system and trigger a systemic inflammatory response syndrome (SIRS).

Systemic inflammatory response syndrome (SIRS) is characterized by increased inflammation (relative to anti-inflammatory processes), increased coagulation (relative to anti-coagulant processes), and decreased fibrinolysis (Beutler B. (2001) Biochem Soc Trans 29:853-9; Bochud P Y. et al. (2003) J Immunol 170:3451-4; Kang T J. et al. (2002) Cytokine 20:56-62; Knaus W A. et al. (1991) Chest 100: 1619-36; Lorenz E. et al. (2000) Infect Immun 68:6398-401, Sorensen T I. et al. (1988) N Engl J Med 318:727-32). Toll-like receptor 2 (TLR-2) is an innate immunity pattern recognition receptor for peptidoglycan, and has an important role in initiating the host immune response to gram-positive bacteria (Beutler B. (2001) Biochem Soc Trans 29:853-9). TLR-2 binding with peptidoglycan from gram-positive bacteria cell walls initiates intracellular signaling responses that result in the activation of Nuclear Factor Kappa B (NFkappaB) and the induction of pro-inflammatory cytokines. A polymorphism resulting in a tryptophan being exchanged for an arginine at codon 677 of the TLR-2 transcript has been associated with susceptibility to lepromatous leprosy, and with decreased activation of NFkappaB in response to Mycobacterium leprae and decreased serum levels of IL-12 (Bochud P Y. et al. (2003) J Immunol 170:3451-4; Kang T J. et al. (2002) Cytokine 20:56-62). A second polymorphism that results in an Arginine being replaced with a glycine at codon 753 has been associated with decreased TLR-2 responsiveness to bacterial peptides from Borrelia burgdorferi and Treponema pallidum, and with susceptibility to staphylococcal infections in a septic shock population (Lorenz E. et al. (2000) Infect Immun 68:6398-401).

Septic and non-septic stimuli such as bacterial endotoxin and cardiopulmonary bypass (CPB), are known to activate the coagulation system and trigger a systemic inflammatory response syndrome (SIRS).

The human TLR-2 sequence maps to chromosome 4 and extends over 2.6 kb. A representative Homo sapiens TLR-2 mRNA sequence is listed in GenBank under accession number NM 003264 (2621 bp). Furthermore, a TLR-2 sequence (SEQ ID NO:1) is found upstream of the TLR-2 transcriptional start site and is listed in GenBank under dbSNP accession number rs4696480. A SNP is located within the TLR-2 sequence represented by SEQ ID NO:1 at position 201 which corresponds to -16934 relative to the TLR-2 transcriptional start site. This same polymorphic site was previously identified as -16933 (Sutherland A M. et al. Crit Care Med. (2005) 33(3): specific pages unknown), but has subsequently been renumbered. An alternative reference number for SNP-16934 is IIPGA-TLR2_{—}540 (www.innateimmunity.net/IIPGA/IIPGASNPs SNP information was retrieved from the Innate Immunity PGA, NHLBI Program in Genomic Applications. Riva A. and Kohane I S. A Web-Based Tool to Retrieve Human Genome Polymorphisms from Public Databases AMIA 2001 Annual conference, Washington D.C., November 2001).

### SUMMARY OF THE INVENTION

This invention is based in part on the surprising discovery that particular single nucleotide polymorphisms (SNPs) from the human toll-like receptor 2 (TLR-2) sequence can be a predictor of subject outcome from an inflammatory condition.

This invention is based in part on the surprising discovery of a TLR-2 SNP that is associated with improved prognosis or subject outcome, in subjects with an inflammatory condition. Furthermore, a TLR-2 SNP is provided which is useful for subject screening, as an indication of subject outcome, or for prognosis for recovery from an inflammatory condition.

This invention is also based in part on the identification the particular nucleotide at the site of a given SNP which is associated with a decreased likelihood of recovery from an inflammatory condition (i.e. ‘risk genotype’) or an increased likelihood of recovery from an inflammatory condition (i.e. ‘protective genotype’).

In accordance with one aspect of the invention, methods are provided for obtaining a prognosis or predicting ability to recover for a subject having or at risk of developing an inflammatory condition, the method including determining a genotype of the subject which includes one or more polymorphic sites in the subject's TLR-2 sequence, wherein the genotype is indicative of an ability of the subject to recover from the inflammatory condition.

In accordance with another aspect of the invention, methods are provided for obtaining a prognosis or predicting ability to recover for a subject having or at risk of developing an inflammatory condition, the method including the step of determining a genotype of the subject which includes one or more polymorphic sites in the subject's TLR-2 sequence, wherein the genotype is indicative of an ability of the subject to recover from the inflammatory condition. The method may further include the step of obtaining the subject's genetic sequence information prior to determining the genotype for a subject and furthermore the method may include the step of obtaining a biological sample from the subject containing genetic sequence information. Additionally, the method may comprise identifying a patient at risk of or having an inflammatory condition.

In accordance with another aspect of the invention, methods are provided for obtaining a prognosis or predicting ability to recover for a subject having or at risk of developing an inflammatory condition, the method may including any one or more of the following steps:
(a) identifying a patient at risk of or having an inflammatory condition;
(b) obtaining a biological sample from the subject;
(c) obtaining the subject's genetic sequence information;
(d) determining a genotype of the subject which includes one or more polymorphic sites in the subject's TLR-2 sequence;
   wherein the genotype is indicative of an ability of the subject to recover from the inflammatory condition.

The polymorphic site may be at SNP 201 (position 201 of SEQ ID NO:1) or at a polymorphic site in linkage disequilibrium thereto. The polymorphic site in linkage disequilibrium with SNP-16934 may have a D′ value of ≧0.5 (or r² value≧0.5). The method may further include comparing the genotype determined with known genotypes which are known to be indicative of a prognosis for recovery from: (i) the subject's type of inflammatory condition; or (ii) another inflammatory condition. The method may further include determining the TLR-2 sequence information for the subject and the method may further include determining the genotype from a nucleic acid sample obtained from the subject. Determining of genotype may include one or more of the following: restriction fragment length analysis; sequencing; hybridization; oligonucleotide ligation assay; ligation rolling circle amplification; 5′ nuclease assay; polymerase proofreading methods; allele specific PCR; and reading sequence data.

A risk genotype of the subject may be indicative of a decreased likelihood of recovery from an inflammatory condition or an increased risk of having a poor outcome. Risk genotype where the subject is critically ill may be indicative of a prognosis of severe cardiovascular or respiratory dysfunction. The risk genotype may include at least one T nucleotide at position 201 of SEQ ID NO:1.

A protective genotype of the subject may be indicative of an increased likelihood of recovery from an inflammatory condition. Where the subject is critically ill the protective genotype may be indicative of a prognosis of less severe cardiovascular or respiratory dysfunction. The protective genotype may be homozygous for the A nucleotide at position 201 of SEQ ID NO:1.

In accordance with another aspect of the invention, methods are provided for identifying a polymorphism in a TLR-2 sequence that correlates with prognosis of recovery from an inflammatory condition in a subject, the method including:
(a) obtaining TLR-2 sequence information from a group of subjects with an inflammatory condition;
(b) identifying at least one polymorphic nucleotide position in the TLR-2 sequence in the subjects;
(c) determining a genotype at the polymorphic site for individual subjects in the group;
(d) determining recovery capabilities of individual subjects in the group from the inflammatory condition; and
(e) correlating genotypes determined in step (c) with the recovery capabilities determined in step (d)
thereby identifying said TLR-2 polymorphisms that correlate with recovery.

The inflammatory condition may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for patients undergoing major surgery or dialysis, patients who are immunocompromised, patients on immunosuppressive agents, patients with HIV/AIDS, patients with suspected endocarditis, patients with fever, patients with fever of unknown origin, patients with cystic fibrosis, patients with diabetes mellitus, patients with chronic renal failure, patients with bronchiectasis, patients with chronic obstructive lung disease, chronic bronchitis, emphysema, or asthma, patients with febrile neutropenia, patients with meningitis, patients with septic arthritis, patients with urinary tract infection, patients with necrotizing fasciitis, patients with other suspected Group A streptococcus infection, patients who have had a splenectomy, patients with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

The determining of a genotype may be accomplished by any technique known in the art, including but not limited to one or more of: restriction fragment length analysis; sequencing; hybridization; oligonucleotide ligation assay; ligation rolling circle amplification; 5′ nuclease assay; polymerase proofreading methods; allele specific PCR; matrix assisted laser desorption ionization time of flight MALDI-TOF mass spectroscopy micro-sequencing assay; gene chip hybridization assays; and reading sequence data.

In accordance with another aspect of the invention, there is provided a kit for determining a genotype at a defined nucleotide position within a polymorphism site in a TLR-2 sequence from a subject to provide a prognosis of the subject's ability to recover from an inflammatory condition, the kit comprising, a restriction enzyme capable of distinguishing alternate nucleotides at the polymorphism site or a labeled oligonucleotide having sufficient complementarity to the polymorphism site and capable of distinguishing said alternate nucleotides. The kit may also include one or more of the following: a package; instructions for using the kit to determine genotype; reagents such a buffers, nucleotides and enzymes. A kit as described herein may contain any combination of the following: a restriction enzyme capable of distinguishing alternate nucleotides at a TLR-2 polymorphism site; and/or a labeled oligonucleotide having sufficient complementary to the TLR-2 polymorphism site and capable of distinguishing said alternate nucleotides; and/or an oligonucleotide or a set of oligonucleotides suitable for amplifying a region including the TLR-2 polymorphism site. The kit may also include one or more of the following: a package; instructions for using the kit to determine genotype; reagents such a buffers, nucleotides and enzymes; and/or containers.

The kit comprising a restriction enzyme may also comprise an oligonucleotide or a set of oligonucleotides suitable to amplify a region surrounding the polymorphism site, a polymerization agent and instructions for using the kit to determine genotype.

In accordance with another aspect of the invention, there is provided a kit for determining a genotype at a defined nucleotide position within a polymorphism site in a TLR-2 sequence from a subject to provide a prognosis of the subject's ability to recover from an inflammatory condition, the kit comprising, in a package a restriction enzyme capable of distinguishing alternate nucleotides at the polymorphism site or a labeled oligonucleotide having sufficient complementary to the polymorphism site and capable of distinguishing said alternate nucleotides. The polymorphism site may correspond to position 201 of SEQ ID NO:1 or position 540 of SEQ ID NO:2.

In accordance with another aspect of the invention, oligonucleotides are provided that may be used in the identification of TLR-2 polymorphisms in accordance with the methods described herein, the oligonucleotides are characterized in that the oligonucleotides hybridize under normal hybridization conditions with a region of one of sequences identified by SEQ ID NO:1 or its complement.

In accordance with another aspect of the invention, an oligonucleotide primer is provided comprising a portion of SEQ ID NO:1, or its complement, wherein said primer is ten to fifty-four nucleotides in length and wherein the primer specifically hybridizes to a region of SEQ ID NO:1 or its complement and is capable of specifically identifying TLR-2 polymorphisms described herein. Alternatively, the primers may be between sixteen to twenty-four nucleotides in length.

In accordance with another aspect of the invention, methods are provided for subject screening, comprising the steps of (a) obtaining TLR-2 sequence information from a subject, and (b) determining the identity of one or more polymorphisms in the sequence, wherein the one or more polymorphisms may be indicative of the ability of a subject to recover from an inflammatory condition.

In accordance with another aspect of the invention methods are provided for subject screening whereby the method includes the steps of (a) selecting a subject based on risk of developing an inflammatory condition or having an inflammatory condition, (b) obtaining TLR-2 sequence information from the subject and (c) detecting the identity of one or more polymorphisms in the sequence, wherein the polymorphism is indicative of the ability of a subject to recover from an inflammatory condition.

In accordance with another aspect of the invention, methods are provided for selecting a group of subjects to determine the efficacy of a candidate drug known or suspected of being useful for the treatment of an inflammatory condition, the method including determining a genotype for one or more polymorphism sites in the TLR-2 sequence for each subject, wherein said genotype is indicative of the subject's ability to recover from the inflammatory condition and sorting subjects based on their genotype. The method may also include administering the candidate drug to the subjects or a subset of subjects and determining each subject's ability to recover from the inflammatory condition. The method may also include the additional step of comparing subject response to the candidate drug based on genotype of the subject. Response to the candidate drug may be decided by determining each subject's ability to recover from the inflammatory condition.

Risk genotype may have at least one T nucleotide at position 201 of SEQ ID NO:1 and may also be homozygous for T at position 201 of SEQ ID NO:1.

Risk genotype may be an indication of an increased risk of not recovering from an inflammatory condition.

Non-risk genotypes or protective genotypes may have be homozygous for A at position 201 of SEQ ID NO:1.

In accordance with another aspect of the invention, methods are provided for selecting a group of subjects for determining the efficacy of a candidate drug known or suspected of being useful for the treatment of a gram positive infection, the method including determining a genotype for one or more polymorphic sites in the TLR-2 sequence for each subject, wherein the genotype is indicative of the subject's likelihood of developing a gram positive infection and sorting subjects based on their genotype.

The method may also include administering the candidate drug to the subjects or a subset of subjects and determining each subject's ability to recover from the gram positive infection. The method may further include comparing subject response to the candidate drug based on genotype of the subject.

In accordance with another aspect of the invention, methods are provided for treating a gram positive infection in a subject in need thereof, the method including administering to the subject an antibiotic agent, wherein said subject has a TLR-2 risk genotype.

In accordance with another aspect of the invention, methods are provided for treating a gram positive infection in a subject in need thereof, the method including selecting a subject having a risk genotype in their TLR-2 sequence and administering to the subject an antibiotic agent.

In accordance with another aspect of the invention, methods are provided for treating a gram positive infection with an antibiotic agent, including identifying a subject having a TLR-2 risk genotype, wherein the identification of a subject with the TLR-2 risk genotype is predictive of an increased likelihood of gram positive infection.

The antibiotic agent may be a gram positive specific antibiotic agent and may further be selected from the following: linezolid (Zyvox®); cloxicillin; methecillin; nafcillin; oxacillin; vancomycin; tazobacam; imipenem; carbenem; meropenem; clindamycin; rifampin; a cephalosporin; a macrolide; quinupristin-dalfoprisin; trimethoprim-sulfamethaxazol; rifampin; amoxicillin; a penicillin; gentamicin; ceftriaxone; ampicillin; cefotaxime; doxycycline; ciprofloxacin; erythromycin and metronidazole.

In accordance with another aspect of the invention, uses of an antibiotic agent are provided for in manufacture of a medicament for the treatment of a gram positive infection, wherein the subjects treated have a TLR-2 risk genotype.

In accordance with another aspect of the invention, uses of an antibiotic agent in the manufacture of a medicament for the treatment of gram positive infection in a subset of subjects, wherein the subset of subjects have a TLR-2 risk genotype.

In accordance with another aspect of the invention, methods are provided determining a risk of developing a gram positive infection in a subject, the method including determining a genotype of the subject at a polymorphic site in the subject's toll-like receptor 2 (TLR-2) sequence, wherein said genotype is indicative the subject's risk of gram positive infection.

The method may further include determining the TLR-2 sequence information for the subject and such a determinion of genotype may be performed on a nucleic acid sample from the subject. The method may further include obtaining a nucleic acid sample from the patient. The genotype of the subject is indicative of a subject's risk of developing a gram positive infection and the TLR-2 risk genotype may be at position 201 of SEQ ID NO:1 and the TLR-2 risk genotype may have at least one A nucleotide at position 201 of SEQ ID NO:1. Conversely, the TLR-2 protective genotype may be homozygous for the T nucleotide at position 201 of SEQ ID NO:1.

In accordance with another aspect of the invention, there is provided an oligonucleotide of about 10 to about 400 nucleotides that hybridizes specifically to a sequence contained in a human target sequence including of SEQ ID NO:1 and SEQ ID NO:2, a complementary sequence of the target sequence or RNA equivalent of the target sequence and wherein the oligonucleotide is operable in determining the TLR-2 sequence polymorphism genotype. The genotype may be a risk or protective genotype.

In accordance with another aspect of the invention, there is provided an oligonucleotide of about 10 to about 400 nucleotides that hybridizes specifically to a sequence contained in a human target sequence including of SEQ ID NO:1 and SEQ ID NO:2, a complementary sequence of the target sequence or RNA equivalent of the target sequence and wherein said hybridization is operable in determining the TLR-2 sequence polymorphism genotype. The genotype may be a risk or protective genotype.

In accordance with another aspect of the invention, there is provided an oligonucleotide probe selected from the group including:
(a) a probe that hybridizes under high stringency conditions to a nucleic acid molecule including SEQ ID NO:1 having a A at position 201 but not to a nucleic acid molecule including SEQ ID NO:1 having a T at position 201; and
(b) a probe that hybridizes under high stringency conditions to a nucleic acid molecule including SEQ ID NO:1 having a T at position 201 but not to a nucleic acid molecule including SEQ ID NO:1 having a A at position 201.

In accordance with another aspect of the invention, there is provided an array of nucleic acid molecules attached to a solid support, the array including an oligonucleotide that will hybridize to a nucleic acid molecule consisting of SEQ ID NO:1, wherein the nucleotide at position 201 is A, under conditions in which the oligonucleotide will not substantially hybridize to a nucleic acid molecule consisting of SEQ ID NO:1 wherein the nucleotide at position 201 is T.

In accordance with another aspect of the invention, there is provided an array of nucleic acid molecules attached to a solid support, the array including an oligonucleotide that will hybridize to a nucleic acid molecule consisting of SEQ ID NO:1, wherein the nucleotide at position 201 is T, under conditions in which the oligonucleotide will not substantially hybridize to a nucleic acid molecule consisting of SEQ ID NO:1 wherein the nucleotide at position 201 is A.

The oligonucleotides may further include one or more of the following: a detectable label; a quencher; a mobility modifier; a contiguous non-target sequence situated 5′ or 3′ to the target sequence.

In accordance with another aspect of the invention, there is provided a computer readable medium including a plurality of digitally encoded genotype correlations selected from the TLR-2 genotype correlations in TABLE 1B, wherein each correlation of the plurality has a value representing an ability to recover from an inflammatory condition or the susceptibility to a gram positive infection.

The above identified sequence positions refer to the sense strand of the TLR-2 sequence as indicated. It will be obvious to a person skilled in the art that analysis could be conducted on the anti-sense strand to determine subject outcome.

### BRIEF DESCRIPTION OF THE DRAWINGS

FIG. 1 shows haplotypes and haplotype clades for toll-like receptor 2 (TLR-2)

FIG. 2 shows an unrooted phylogenetic tree for TLR-2 haplotype clades.

FIG. 3 shows a Kaplan Meier survival curve over 28 days for patients with TLR-2-16934 AA, TLR-2-16934 AT, and TLR-2-16934 TT.

FIG. 4 shows a graph of an association between TLR-2-16934 genotype and frequency of sepsis on admission to the ICU.

FIG. 5 shows a graph of an association between TLR-2-16934 T/A genotype and days alive and free of cardiovascular dysfunction, days alive and free of vasopressors and days alive and free of inotropic agents.

FIG. 6 shows a graph of an association between the TLR-2-16934 T/A genotype and 3/4 SIRS criteria and days alive and free of steroid use.

FIG. 7 shows a graph of an association between TLR2-16934 A/T genotype and days alive and free of coagulation and between TLR2-16934 A/T genotype and days alive and free of INR>1.5.

FIG. 8 shows a graph of an association between TLR-2-16934 A/T genotype and days alive and free of renal support and between TLR-2-16934 A/T genotype and days alive and free of hepatic dysfunction.

FIG. 9 shows a Kaplan Meier survival curve over 28 days of patients who were TLR-2-16934 AA, TLR-2-16934 AT, and TLR-2-16934 TT.

FIG. 10 shows a graph of an association between TLR-2-16934 T/A genotype with days alive and free of days alive and free of vasopressors and cardiovascular dysfunction in critically ill patients with sepsis.

FIG. 11 shows a graph of an association between TLR-2-16934 T/A genotype with days alive and free of 3/4 SIRS criteria and days alive and free of steroid support in critically ill patients with sepsis.

FIG. 12 shows a graph of an association between TLR-2-16934 A/T genotype and days alive and free of coagulation, days alive and free of INR>1.5 and days alive and free of hepatic dysfunction in critically ill patients with sepsis.

FIG. 13 shows a graph of an association between TLR-2-16934 A/T genotype and days alive and free of respiratory dysfunction and days alive and free of mechanical ventilation in critically ill patients with sepsis.

### DETAILED DESCRIPTION OF THE INVENTION

### 1. Definitions

In the description that follows, a number of terms are used extensively, the following definitions are provided to facilitate understanding of the invention.

“Genetic material” includes any nucleic acid and can be a deoxyribonucleotide or ribonucleotide polymer in either single or double-stranded form.

A “purine” is a heterocyclic organic compound containing fused pyrimidine and imidazole rings, and acts as the parent compound for purine bases, adenine (A) and guanine (G). “Nucleotides” are generally a purine (R) or pyrimidine (Y) base covalently linked to a pentose, usually ribose or deoxyribose, where the sugar carries one or more phosphate groups. Nucleic acids are generally a polymer of nucleotides joined by 3′-5′ phosphodiester linkages. As used herein “purine” is used to refer to the purine bases, A and G, and more broadly to include the nucleotide monomers, deoxyadenosine-5′-phosphate and deoxyguanosine-5′-phosphate, as components of a polynucleotide chain.

A “pyrimidine” is a single-ringed, organic base that forms nucleotide bases, cytosine (C), thymine (T) and uracil (U). As used herein “pyrimidine” is used to refer to the pyrimidine bases, C, T and U, and more broadly to include the pyrimidine nucleotide monomers that along with purine nucleotides are the components of a polynucleotide chain.

A nucleotide represented by the symbol M may be either an A or C, a nucleotide represented by the symbol W may be either an T or A, a nucleotide represented by the symbol Y may be either an C or T, a nucleotide represented by the symbol S may be either an G or C, while a nucleotide represented by the symbol R may be either an G or A.

A “polymorphic site” or “polymorphism site” or “polymorphism” or “single nucleotide polymorphism site” (SNP site) as used herein is the locus or position within a given sequence at which divergence occurs. A “Polymorphism” is the occurrence of two or more forms of a gene or position within a gene (allele), in a population, in such frequencies that the presence of the rarest of the forms cannot be explained by mutation alone. The implication is that polymorphic alleles confer some selective advantage on the host. Preferred polymorphic sites have at least two alleles, each occurring at frequency of greater than 1%, and more preferably greater than 10% or 20% of a selected population. Polymorphism sites may be at known positions within a nucleic acid sequence or may be determined to exist using the methods described herein. Polymorphisms may occur in both the coding regions and the noncoding regions (for example, promoters, enhancers and introns) of genes.

In general the term “linkage”, as used in population genetics, refers to the co-inheritance of two or more nonallelic genes due to the close proximity of the loci on the same chromosome, whereby after meiosis they remain associated more often than the 50% expected for unlinked genes. However, during meiosis, a physical crossing between individual chromatids may result in recombination. “Recombination” generally occurs between large segments of DNA, whereby contiguous stretches of DNA and genes are likely to be moved together in the recombination event (crossover). Conversely, regions of the DNA that are far apart on a given chromosome are more likely to become separated during the process of crossing-over than regions of the DNA that are close together. Polymorphic molecular markers, like single nucleotide polymorphisms (SNPs), are often useful in tracking meiotic recombination events as positional markers on chromosomes.

A “risk genotype” as used herein refers to an allelic variant (genotype) at one or more polymorphism sites within the TLR-2 sequence described herein as being indicative of a decreased likelihood of recovery from an inflammatory condition or an increased risk of having a poor outcome. The risk genotype may be determined for either the haploid genotype or diploid genotype, provided that at least one copy of a risk allele is present. Such “risk alleles” or “risk genotype” may be a T nucleotide at SNP -16934 (position 201 of SEQ ID NO:1) (toll-like receptor 2). Risk genotype may be an indication of an increased risk of not recovering from an inflammatory condition. Subjects having one copy (heterozygotes) or two copies (homozygotes) of the risk allele are considered to have the “risk genotype” even though the degree to which the subjects risk of not recovering from an inflammatory condition increases, may be greater for homozygotes over heterozygotes.

A “clade” is a group of haplotypes that are closely related phylogenetically. For example, if haplotypes are displayed on a phylogenetic (evolutionary) tree a clade includes all haplotypes contained within the same branch.

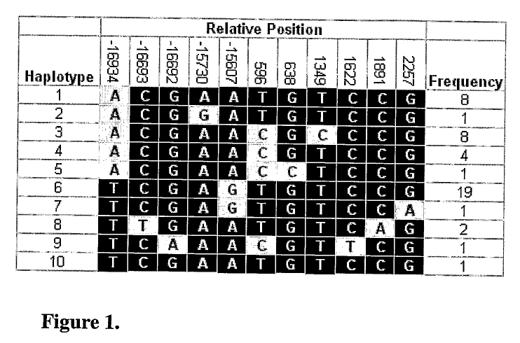

As used herein “haplotype” is a set of alleles of closely linked loci or a pattern of a set of markers along a chromosome that tend to be inherited together. Accordingly, groups of alleles on the same small chromosomal segment tend to be transmitted together. Haplotypes along a given segment of a chromosome are generally transmitted to progeny together unless there has been a recombination event. Absent a recombination event, haplotypes can be treated as alleles at a single highly polymorphic locus for mapping. “Haplotypes” are shown as rows in the Table (haplotype map) represented in FIG. 1.

In general, the detection of nucleic acids in a sample and the subtypes thereof depends on the technique of specific nucleic acid hybridization in which the oligonucleotide probe is annealed under conditions of “high stringency” to nucleic acids in the sample, and the successfully annealed probes are subsequently detected (Spiegelman, S., Scientific American, Vol. 210, p. 48 (1964)). Hybridization under high stringency conditions primarily depends on the method used for hybridization. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

As used herein “linkage disequilibrium” (LD) is the occurrence in a population of certain combinations of linked alleles in greater proportion than expected from the allele frequencies at the loci. For example, the preferential occurrence of a disease gene in association with specific alleles of linked markers, such as SNPs, or between specific alleles of linked markers, are considered to be in LD. This sort of disequilibrium generally implies that most of the disease chromosomes carry the same mutation and that the markers being tested are relatively close to the disease gene(s). Accordingly, if the genotype of a first locus is in LD with a second locus (or third locus etc.), the determination of the allele at only one locus would necessarily provide the identity of the allele at the other locus. When evaluating loci for LD those sites within a given population having a high degree of linkage disequilibrium (i.e. an absolute value for D′ of ≧0.5 or r²≧0.5) are potentially useful in predicting the identity of an allele of interest (i.e. associated with the condition of interest). Alternatively, a high degree of linkage disequilibrium may be represented by an absolute value for D′ of ≧0.7 or r²≧0.7 or by an absolute value for D′ of ≧0.8 or r²≧0.8. Accordingly, two SNPs that have a high degree of LD may be equally useful in determining the identity of the allele of interest or disease allele. Therefore, we may assume that knowing the identity of the allele at one SNP may be representative of the allele identity at another SNP in LD. Accordingly, the determination of the genotype of a single locus can provide the identity of the genotype of any locus in LD therewith and the higher the degree of linkage disequilibrium the more likely that two SNPs may be used interchangeably.

Additional sites may be identified as polymorphism sites in the TLR-2 sequence, where those polymorphisms are linked to the polymorphism at position SNP -16934 (position 201 of SEQ ID NO:1) and may also therefore be indicative of a subject's prognosis.

It will be appreciated by a person of skill in the art that further linked SNP sites could be determined. The haplotype for TLR-2 can be created by assessing the SNPs of the TLR-2 sequence or TLR-2 sequence in normal subjects using a program that has an expectation maximization algorithm (i.e. PHASE). A constructed haplotype of TLR-2 may be used to find combinations of SNP's that are in linkage disequilibrium (LD) with SNP -16934 (position 201 of SEQ ID NO:1). Therefore, the haplotype of an individual could be determined by genotyping other SNPs that are in LD with SNP -16934 (position 201 of SEQ ID NO:1).

It will be appreciated by a person of skill in the art, that the numerical designations of the positions of polymorphisms within a sequence are relative to a specific sequence and that the same positions may be assigned different numerical designations depending on the way in which the sequence is numbered and the sequence chosen, as illustrated by the alternative numbering of equivalent polymorphisms in TABLE 1A below. Furthermore, sequence variations within the population, such as insertions or deletions, may change the relative position and subsequently the numerical designations of particular nucleotides at and around a polymorphism site.

A Homo sapiens toll-like receptor 2 (TLR-2) sequence comprises a sequence as listed in GenBank dbSNP accession number rs4696480 identified herein as SEQ ID NO:1. The major and minor alleles for -16934 or position 201 of SEQ ID NO:1 are as follows: the most common nucleotide (major allele) is a and the minor allele is t.

TABLE 1A below shows the flanking sequences for SNP T-16934A (rs4696480) of TLR-2 along with associated SNP location within the sequence represented by a W at position 201 and within the gene relative to the transcription start site (-16934) for the TLR-2 gene (also corresponding to the W at position 540 of SEQ ID NO:2).

TABLE 1A

| | SNP | |
|---|---|---|
| | locations | |
| TLR-2 | in | |
| SNP | TLR-2 | FLANKING SEQUENCE |
| -16934 | 5′ | Ccccaaattt aaaagagggc aagaaaagag |
| shown at | promoter | agacaataga acataaaaca aatgggacaa |
| position | region | gaataaagta catagttgtc acagtccctt |
| 201 of | -16934 | gggtgctgct gtaacaaaat acctgagact |
| SEQ ID | relative | gggtaattta caaagaacag aaatttatcc |
| NO:1 | to | attcatggtt ctggagtctg ggaagtccaa |
| | tran- | gattgaaggg ctgcatctgg Wgagggtcat |
| | scription | ctggctacat tataacatga tggaaagcat |
| | start | cacatggtga gagagagcaa gagaggacag |
| | site | aacttacttt tataacaaac tcattctcac |
| | | aataaactgc tcccttggtg ataacattaa |
| | | tccatttatg agggtacagc cttcataacc |
| | | taatcacctc ttaaaaggtt ccacctctca |
| | | gctcgcagtg a |

The Sequences given in TABLE 1A above and in SEQ ID NO:2 would be useful to a person of skill in the art in the design of primers and probes or other oligonucleotides for the identification of TLR-2 SNP alleles and or genotypes as described herein.

TABLE 1B below shows genotype correlations for TLR-2 SNPs with a value representing an ability to recover from an inflammatory condition or predicted patient outcome wherein the allele is additive producing an intermediate value for the heterozygote. However, it will be appreciated by persons of skill in the art that the Inflammatory Condition Patient Score or Gram+Infection Susceptibility Patient Score may have a dominant/recessive relationship whereby the heterozygote provides the same score as one of the homozygotes. The relationship may also depend on the population tested.

TABLE 1B

| Position in | | | Inflammatory | Gram + Infection |
|---|---|---|---|---|
| SEQ ID | | | Condition Patient | Susceptibility |
| NO: 1 | Allele | Genotype | Score* | Patient Score* |
| 201 | A | AA | 2 | 0 |
| 201 | A/T | AT | 1 | 1 |
| 201 | T | TT | 0 | 2 |

*good = 2; moderate = 1; poor = 0.

An “allele” is defined as any one or more alternative forms of a given gene at a particular locus on a chromosome. Different alleles produce variation in inherited characteristics such as hair color or blood type. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene. In an individual, one form of the allele (major) may be expressed more than another form (minor). When “genes” are considered simply as segments of a nucleotide sequence, allele refers to each of the possible alternative nucleotides at a specific position in the sequence. For example, a CT polymorphism such as CCT[C/T]CCAT would have two alleles: C and T.

A “gene” is an ordered sequence of nucleotides located in a particular position on a particular chromosome that encodes a specific functional product and may include untranslated and untranscribed sequences in proximity to the coding regions. Such non-coding sequences may contain regulatory sequences needed for transcription and translation of the sequence or introns etc.

A “genotype” is defined as the genetic constitution of an organism, usually in respect to one gene or a few genes or a region of a gene relevant to a particular context (for example the genetic loci responsible for a particular phenotype). A region of a gene can be as small as a single nucleotide in the case of a single nucleotide polymorphism.

A “phenotype” is defined as the observable characters of an organism.

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A “transition” is the replacement of one purine by another purine or one pyrimidine by another pyrimidine. A “transversion” is the replacement of a purine by a pyrimidine or vice versa. Single nucleotide polymorphisms can also arise from a deletion (represented by “−” or “del”) of a nucleotide or an insertion (represented by “+” or “ins”) of a nucleotide relative to a reference allele. Furthermore, it would be appreciated by a person of skill in the art, that an insertion or deletion within a given sequence could alter the relative position and therefore the position number of another polymorphism within the sequence.

A “systemic inflammatory response syndrome” or (SIRS) is defined as including both septic (i.e. sepsis or septic shock) and non-septic systemic inflammatory response (i.e. post operative). “SIRS” is further defined according to ACCP (American College of Chest Physicians) guidelines as the presence of two or more of A) temperature>38° C. or <36° C., B) heart rate>90 beats per minute, C) respiratory rate>20 breaths per minute, and D) white blood cell count>12,000 per mm3 or <4,000 mm3. In the following description, the presence of two, three, or four of the “SIRS” criteria were scored each day over the 28 day observation period.

“Sepsis” is defined as the presence of at least two “SIRS” criteria and known or suspected source of infection. Septic shock was defined as sepsis plus one new organ failure by Brussels criteria plus need for vasopressor medication.

Patient outcome or prognosis as used herein refers the ability of a patient to recover from an inflammatory condition. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for patients undergoing major surgery or dialysis, patients who are immunocompromised, patients on immunosuppressive agents, patients with HIV/AIDS, patients with suspected endocarditis, patients with fever, patients with fever of unknown origin, patients with cystic fibrosis, patients with diabetes mellitus, patients with chronic renal failure, patients with bronchiectasis, patients with chronic obstructive lung disease, chronic bronchitis, emphysema, or asthma, patients with febrile neutropenia, patients with meningitis, patients with septic arthritis, patients with urinary tract infection, patients with necrotizing fasciitis, patients with other suspected Group A streptococcus infection, patients who have had a splenectomy, patients with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Assessing subject outcome or prognosis may be accomplished by various methods. For Example, an “APACHE II” score is defined as Acute Physiology And Chronic Health Evaluation and herein was calculated on a daily basis from raw clinical and laboratory variables. Vincent et al. (Vincent J L. Ferreira F. Moreno R. Scoring systems for assessing organ dysfunction and survival. Critical Care Clinics. 16:353-366, 2000) summarize APACHE score as follows “First developed in 1981 by Knaus et al., the APACHE score has become the most commonly used survival prediction model in ICUs worldwide. The APACHE II score, a revised and simplified version of the original prototype, uses a point score based on initial values of 12 routine physiologic measures, age, and previous health status to provide a general measure of severity of disease. The values recorded are the worst values taken during the subject's first 24 hours in the ICU. The score is applied to one of 34 admission diagnoses to estimate a disease-specific probability of mortality (APACHE H predicted risk of death). The maximum possible APACHE II score is 71, and high scores have been well correlated with mortality. The APACHE II score has been widely used to stratify and compare various groups of critically ill subjects, including subjects with sepsis, by severity of illness on entry into clinical trials.”

A “Brussels score” score is a method for evaluating organ dysfunction as compared to a baseline. If the Brussels score is 0 (i.e. moderate, severe, or extreme), then organ failure was recorded as present on that particular day (see TABLE 2A below). In the following description, to correct for deaths during the observation period, days alive and free of organ failure (DAF) were calculated as previously described. For example, acute lung injury was calculated as follows. Acute lung injury is defined as present when a subject meets all of these four criteria. 1) Need for mechanical ventilation, 2) Bilateral pulmonary infiltrates on chest X-ray consistent with acute lung injury, 3) PaO₂/FiO₂ ratio is less than 300, 4) No clinical evidence of congestive heart failure or if a pulmonary artery catheter is in place for clinical purposes, a pulmonary capillary wedge pressure less than 18 mm Hg. The severity of acute lung injury is assessed by measuring days alive and free of acute lung injury over a 28 day observation period. Acute lung injury is recorded as present on each day that the person has moderate, severe or extreme dysfunction as defined in the Brussels score. Days alive and free of acute lung injury is calculated as the number of days after onset of acute lung injury that a subject is alive and free of acute lung injury over a defined observation period (28 days). Thus, a lower score for days alive and free of acute lung injury indicates more severe acute lung injury. The reason that days alive and free of acute lung injury is preferable to simply presence or absence of acute lung injury, is that acute lung injury has a high acute mortality and early death (within 28 days) precludes calculation of the presence or absence of acute lung injury in dead subjects. The cardiovascular, renal, neurologic, hepatic and coagulation dysfunction were similarly defined as present on each day that the person had moderate, severe or extreme dysfunction as defined by the Brussels score. Days alive and free of steroids are days that a person is alive and is not being treated with exogenous corticosteroids (e.g. hydrocortisone, prednisone, methylprednisolone). Days alive and free of pressors are days that a person is alive and not being treated with intravenous vasopressors (e.g. dopamine, norepinephrine, epinephrine, phenylephrine). Days alive and free of an International Normalized Ratio (INR)>1.5 are days that a person is alive and does not have an INR>1.5.

TABLE 2A
Brussels Organ Dysfunction Scoring System

| | Free of Organ | | Clinically Significant | | |
|---|---|---|---|---|---|
| | Dysfunction | | Organ Dysfunction | | |
| | Normal | Mild | Moderate | Severe | Extreme |
| | DAF ORGAN DYSFUNCTION | | | | |
| | SCORE | | | | |
| ORGANS | 1 | | 0 | | |
| Cardio- | >90 | ≦90 | ≦90 | ≦90 plus | ≦90 plus |
| vascular | | Respon- | Unresponsive | pH ≦ 7.3 | pH ≦ 7.2 |
| Systolic BP | | sive | to fluid | | |
| (mmHg) | | to fluid | | | |
| Pulmonary | >400 | 400-301 | 300-201 | 200-101 | ≦100 |
| Pₐo₂/Fₗo₂ | | | Acute lung | ARDS | Severe |
| (mmHg) | | | injury | | ARDS |
| Renal | <1.5 | 1.5-1.9 | 2.0-3.4 | 3.5-4.9 | ≧5.0 |
| Creatinine | | | | | |
| (mg/dL) | | | | | |
| Hepatic | <1.2 | 1.2-1.9 | 2.0-5.9 | 6.0-11.9 | ≧12 |
| Bilirubin | | | | | |
| (mg/dL) | | | | | |
| Hematologic | >120 | 120-81 | 80-51 | 50-21 | ≦20 |
| Platelets | | | | | |
| (×10⁵/mm³) | | | | | |
| Neurologic | 15 | 14-13 | 12-10 | 9-6 | ≦5 |
| (Glascow | | | | | |
| Score) | | | | | |

Round Table Conference on Clinical Trials for the Treatment of Sepsis Brussels, Mar. 12-14, 1994.

Analysis of variance (ANOVA) is a standard statistical approach to test for statistically significant differences between sets of measurements.

The Fisher exact test is a standard statistical approach to test for statistically significant differences between rates and proportions of characteristics measured in different groups.

### 2. General Methods

One aspect of the invention may involve the identification of subjects or the selection of subjects that are either at risk of developing and inflammatory condition or the identification of subjects who already have an inflammatory condition. For example, subjects who have undergone major surgery or scheduled for or contemplating major surgery may be considered as being at risk of developing an inflammatory condition. Furthermore, subjects may be determined as having an inflammatory condition using diagnostic methods and clinical evaluations known in the medical arts. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for patients undergoing major surgery or dialysis, patients who are immunocompromised, patients on immunosuppressive agents, patients with HIV/AIDS, patients with suspected endocarditis, patients with fever, patients with fever of unknown origin, patients with cystic fibrosis, patients with diabetes mellitus, patients with chronic renal failure, patients with bronchiectasis, patients with chronic obstructive lung disease, chronic bronchitis, emphysema, or asthma, patients with febrile neutropenia, patients with meningitis, patients with septic arthritis, patients with urinary tract infection, patients with necrotizing fascitis, patients with other suspected Group A streptococcus infection, patients who have had a splenectomy, patients with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Once a subject is identified as being at risk for developing or having an inflammatory condition, then genetic sequence information may be obtained from the subject. Or alternatively genetic sequence information may already have been obtained from the subject. For example, a subject may have already provided a biological sample for other purposes or may have even had their genetic sequence determined in whole or in part and stored for future use. Genetic sequence information may be obtained in numerous different ways and may involve the collection of a biological sample that contains genetic material. Particularly, genetic material, containing the sequence or sequences of interest. Many methods are known in the art for collecting bodily samples and extracting genetic material from those samples. Genetic material can be extracted from blood, tissue and hair and other samples. There are many known methods for the separate isolation of DNA and RNA from biological material. Typically, DNA may be isolated from a biological sample when first the sample is lysed and then the DNA is isolated from the lysate according to any one of a variety of multi-step protocols, which can take varying lengths of time. DNA isolation methods may involve the use of phenol (Sambrook, J. et al., “Molecular Cloning”, Vol. 2, pp. 9.14-9.23, Cold Spring Harbor Laboratory Press (1989) and Ausubel, Frederick M. et al., “Current Protocols in Molecular Biology”, Vol. 1, pp. 2.2.1-2.4.5, John Wiley & Sons, Inc. (1994)). Typically, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in Van Ness et al. (U.S. Pat. No. 5,130,423), non-corrosive phenol derivatives are used for the isolation of nucleic acids. The resulting preparation is a mix of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The final purification of the precipitated, crude DNA fraction can be achieved by any one of several methods, including column chromatography (Analects, (1994) Vol 22, No. 4, Pharmacia Biotech), or exposure of the crude DNA to a polyanion-containing protein as described in Koller (U.S. Pat. No. 5,128,247).

Yet another method of DNA isolation, which is described by Botwell, D. D. L. (Anal. Biochem. (1987) 162:463-465) involves lysing cells in 6M guanidine hydrochloride, precipitating DNA from the lysate at acid pH by adding 2.5 volumes of ethanol, and washing the DNA with ethanol.

Numerous other methods are known in the art to isolate both RNA and DNA, such as the one described by Chomczynski (U.S. Pat. No. 5,945,515), whereby genetic material can be extracted efficiently in as little as twenty minutes. Evans and Hugh (U.S. Pat. No. 5,989,431) describe methods for isolating DNA using a hollow membrane filter.

Once a subject's genetic sequence information has been obtained from the subject it may then be further analyzed to detect or determine the identity or genotype of one or more polymorphisms in the TLR-2 sequence. Provided that the genetic material obtained, contains the sequence of interest. Particularly, a person may be interested in determining the TLR-2 genotype of a subject of interest, where the genotype includes a nucleotide corresponding to position 201 of SEQ ID NO:1. The sequence of interest may also include other TLR-2 polymorphisms or may also contain some of the sequence surrounding the polymorphism of interest. Detection or determination of a nucleotide identity or the genotype of one or more single nucleotide polymorphism(s) (SNP typing), may be accomplished by any one of a number methods or assays known in the art. Many DNA typing methodologies are useful for allelic discrimination and detection of SNPs. Furthermore, the products of allelic discrimination reactions or assays may be detected by one or more detection methods. The majority of SNP genotyping reactions or assays can be assigned to one of four broad groups (allele specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, allele specific hybridization involves a hybridization probe, which is capable of distinguishing between two DNA targets differing at one nucleotide position by hybridization. Usually probes are designed with the polymorphic base in a central position in the probe sequence, whereby under optimized assay conditions only the perfectly matched probe target hybrids are stable and hybrids with a one base mismatch are unstable. A strategy which couples detection and allelic discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Similarly, primer extension reactions (i.e. mini sequencing, allele specific extensions, or simple PCR amplification) are useful in allelic discrimination reactions. For example, in mini sequencing a primer anneals to its target DNA immediately upstream of the SNP and is extended with a single nucleotide complementary to the polymorphic site. Where the nucleotide is not complementary no extension occurs.

Oligonucleotide ligation assays require two allele specific probes and one common ligation probe per SNP. The common ligation probe hybridizes adjacent to an allele specific probe and when there is a perfect match of the appropriate allele specific probe the ligase joins both allele specific and the common probes. Where there is not a perfect match the ligase is unable to join the allelic specific and common probes.

Alternatively, an invasive cleavage method requires an oligonucleotide called an invader probe and allele specific probes to anneal to the target DNA with an overlap of one nucleotide. When the allele specific probe is complementary to the polymorphic base, overlaps of the 3′ end of the invader oligonucleotide form a structure that is recognized and cleaved by a Flap endonuclease releasing the 5′ arm of the allele specific probe.

It will be appreciated that numerous other methods for allelic discrimination and detection are known in the art and some of which are described in further detail below. It will also be appreciated that reactions such as arrayed primer extension mini sequencing, tag microarrays and allelic specific extension could be performed on a microarray.

SNP typing methods may include but are not limited to the following:
Restriction Fragment Length Polymorphism (RFLP) strategy—An RFLP gel-based analysis can be used to distinguish between alleles at polymorphic sites within a gene. Briefly, a short segment of DNA (typically several hundred base pairs) is amplified by PCR. Where possible, a specific restriction endonuclease is chosen that cuts the short DNA segment when one variant allele is present but does not cut the short DNA segment when the other allele variant is present. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the initial DNA sample had the allele, which could be cut by the chosen restriction endonuclease. In contrast, if only one higher molecular weight band is observed (at the molecular weight of the PCR product) then the initial DNA sample had the allele variant that could not be cut by the chosen restriction endonuclease. Finally, if both the higher molecular weight band and the two lower molecular weight bands are visible then the initial DNA sample contained both alleles, and therefore the subject was heterozygous for this single nucleotide polymorphism;
Sequencing—For example the Maxam-Gilbert technique for sequencing (Maxam A M. and Gilbert W. Proc. Natl. Acad. Sci. USA (1977) 74(4):560-564) involves the specific chemical cleavage of terminally labelled DNA. In this technique four samples of the same labeled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the same one or two of the four nucleotides. In particular, in one sample each fragment ends with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labeling. Another method is the dideoxy method of sequencing was published by Sanger et al. (Sanger et al. Proc. Natl. Acad. Sci. USA (1977) 74(12):5463-5467). The Sanger method relies on enzymatic activity of a DNA polymerase to synthesize sequence-dependent fragments of various lengths. The lengths of the fragments are determined by the random incorporation of dideoxynucleotide base-specific terminators. These fragments can then be separated in a gel as in the Maxam-Gilbert procedure, visualized, and the sequence determined. Numerous improvements have been made to refine the above methods and to automate the sequencing procedures. Similarly, RNA sequencing methods are also known. For example, reverse transcriptase with dideoxy-nucleotides have been used to sequence encephalomyocarditis virus RNA (Zimmern D. and Kaesberg P. Proc. Natl. Acad. Sci. USA (1978) 75(9):4257-4261). Mills D R. and Kramer F R. (Proc. Natl. Acad. Sci. USA (1979) 76(5):2232-2235) describe the use of Q.beta. replicase and the nucleotide analog inosine for sequencing RNA in a chain-termination mechanism. Direct chemical methods for sequencing RNA are also known (Peattie D A. Proc. Natl. Acad. Sci. USA (1979) 76(4):1760-1764). Other methods include those of Donis-Keller et al. (1977, Nucl. Acids Res. 4:2527-2538), Simoncsits A. et al. (Nature (1977) 269(5631):833-836), Axelrod V D. et al. (Nucl. Acids Res. (1978) 5(10):3549-3563), and Kramer F R. and Mills D R. (Proc. Natl. Acad. Sci. USA (1978) 75(11):5334-5338, which are incorporated herein by reference). Nucleic acid sequences can also be read by stimulating the natural fluoresce of a cleaved nucleotide with a laser while the single nucleotide is contained in a fluorescence enhancing matrix (U.S. Pat. No. 5,674,743); In a mini sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP or only ddNTP in the mini sequencing reaction depends on the method chosen for detecting the products;
Hybridization methods for the identification of SNPs are described in the U.S. Pat. Nos. 6,270,961 & 6,025,136;
A template-directed dye-terminator incorporation with fluorescent polarization-detection (TDI-FP) method is described by FREEMAN B D. et al. (J Mol Diagnostics (2002) 4(4):209-215) is described for large scale screening;
Oligonucleotide ligation assay (OLA)—is based on ligation of probe and detector oligonucleotides annealed to a polymerase chain reaction amplicon strand with detection by an enzyme immunoassay (VILLAHERMOSA ML. J Hum Virol (2001) 4(5):238-48; ROMPPANEN E L. Scand J Clin Lab Invest (2001) 61(2):123-9; IANNONE M A. et al. Cytometry (2000) 39(2): 131-40);
Ligation-Rolling Circle Amplification (L-RCA) has also been successfully used for genotyping single nucleotide polymorphisms as described in QI X. et al. Nucleic Acids Res (2001) 29(22):E116;
5′ nuclease assay has also been successfully used for genotyping single nucleotide polymorphisms (AYDIN A. et al. Biotechiniques (2001) (4):920-2, 924, 926-8.).
For example a 5′ exonuclease activity or TaqMan™ assay (Applied Biosystems) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the major allele and the other to the minor allele. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragment. Mismatched probes are displaced without fragmentation. The genotype of a sample is determined by measuring the signal intensity of the two different dyes;
Polymerase proofreading methods are used to determine SNPs identities, as described in WO 0181631;
Detection of single base pair DNA mutations by enzyme-amplified electronic transduction is described in PATOLSKY F et al. Nat Biotech. (2001) 19(3):253-257;
Gene chip technologies are also known for single nucleotide polymorphism discrimination whereby numerous polymorphisms may be tested for simultaneously on a single array (EP 1120646 and Gilles P N. et al. Nat. Biotechnology (1999) 17(4):365-70). For example, one such array based genotyping platform is the microsphere based tag-it high throughput genotyping array (Bortolin S. et al. Clinical Chemistry (2004) 50(11): 2028-36). This method amplifies genomic DNA by PCR followed by allele specific primer extension with universally tagged genotyping primers. The products are then sorted on a Tag-It array and detected using the Luminex xMAP system;
Matrix assisted laser desorption ionization time of flight (MALDI-TOF) mass spectroscopy is also useful in the genotyping single nucleotide polymorphisms through the analysis of microsequencing products (Haff L A. and Smirnov I P. Nucleic Acids Res. (1997) 25(18):3749-50; Haff L A. and Smirnov I P. Genome Res. (1997) 7:378-388; Sun X. et al. Nucleic Acids Res. (2000) 28 e68; Braun A. et al. Clin. Chem. (1997) 43:1151-1158; Little D P. et al. Eur. J. Clin. Chem. Clin. Biochem. (1997) 35:545-548; Fei Z. et al. Nucleic Acids Res. (2000) 26:2827-2828; and Blondal T. et al. Nucleic Acids Res. (2003) 31(24):e155); or
Allele specific PCR methods have also been successfully used for genotyping single nucleotide polymorphisms (Hawkins J R. et al. Hum Mutat (2002) 19(5):543-553).

Alternatively, if a subject's sequence data is already known, then obtaining may involve retrieval of the subjects nucleic acid sequence data from a database, followed by determining or detecting the identity of a nucleic acid or genotype at a polymorphism site by reading the subject's nucleic acid sequence at the polymorphic site.

Once the identity of a polymorphism(s) is determined or detected an indication may be obtained as to subject outcome or prognosis or ability of a subject recover from an inflammatory condition based on the genotype of the polymorphism of interest. In the present application, polymorphisms in toll-like receptor 2 (TLR-2) sequence, are used to obtain a prognosis or to make a determination regarding ability of the subject to recover from the inflammatory condition. Methods for determining a subject's prognosis or for subject screening may be useful to determine the ability of a subject to recover from an inflammatory condition. Alternatively, single polymorphism sites or combined polymorphism sites may be used as an indication of a subject's ability to recover from an inflammatory condition, if they are linked to a polymorphism determined to be indicative of a subject's ability to recover from an inflammatory condition. The method may further comprise comparing the genotype determined for a polymorphism with known genotypes, which are indicative of a prognosis for recovery from the same inflammatory condition as for the subject or another inflammatory condition. Accordingly, a decision regarding the subject's ability to recover may be from an inflammatory condition may be made based on the genotype determined for the polymorphism site.

Once subject outcome or a prognosis is determined, such information may be of interest to physicians and surgeons to assist in deciding between potential treatment options, to help determine the degree to which subjects are monitored and the frequency with which such monitoring occurs. Ultimately, treatment decisions may be made in response to factors, both specific to the subject and based on the experience of the physician or surgeon responsible for a subject's care.

An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, CNS, coagulation [INR>1.5], renal and/or hepatic).

As described above genetic sequence information or genotype information may be obtained from a subject wherein the sequence information contains one or more single nucleotide polymorphism sites in TLR-2 sequence. Also, as previously described the sequence identity of one or more single nucleotide polymorphisms in TLR-2 sequence of one or more subjects may then be detected or determined. Furthermore, subject outcome or prognosis may be assessed as described above, for example the APACHE II scoring system or the Brussels score may be used to assess subject outcome or prognosis by comparing subject scores before and after treatment. Once subject outcome or prognosis has been assessed, subject outcome or prognosis may be correlated with the sequence identity of one or more single nucleotide polymorphism(s). The correlation of subject outcome or prognosis may further include statistical analysis of subject outcome scores and polymorphism(s) for a number of subjects.

Similarly, once a subject's TLR-2 genotype has been determined or detected an indication may be obtained as to subject likelihood of developing a gram positive bacterial infection based on the genotype of the -16934 TLR-2 SNP. In the present application, polymorphisms in toll-like receptor 2 (TLR-2) sequence, are used to obtain a prognosis or to make a determination regarding developing a gram positive bacterial infection. Methods for determining a subject's likelihood of developing a gram positive bacterial infection. The method may further comprise administration of an antibiotic agent, in particular an gram positive specific antibiotic agent. Furthermore, the decision to administer such agents may be made prior to culturing the patient to determine the type of infection (i.e. gram positive or negative). Gram positive specific antibiotic agents may be selected from the following: linezolid (Zyvox®); cloxicillin; methecillin; nafcillin; oxacillin; vancomycin; tazobacam; imipenem; carbenem; meropenem; clindamycin; rifampin; a cephalosporin; a macrolide; quinupristin-dalfoprisin; trimethoprim-sulfamethaxazol; rifampin; amoxicillin; a penicillin; gentamicin; ceftriaxone; ampicillin; cefotaxime; doxycycline; ciprofloxacin; erythromycin and metronidazole. These agents may be administered according to common practice or manufacturers suggested protocols following TLR-2 genotype determination to identify at risk subjects.

### Clinical Phenotype

The primary outcome variable was survival to hospital discharge. Secondary outcome variables were days alive and free of cardiovascular, respiratory, renal, hepatic, hematologic, and neurologic organ system failure as well as days alive and free of SIRS (Systemic Inflammatory Response Syndrome), occurrence of sepsis, and occurrence of septic shock. SIRS was considered present when subjects met at least two of four SIRS criteria. The SIRS criteria were 1) fever (>38° C.) or hypothermia (<35.5° C.), 2) tachycardia (>100 beats/min in the absence of beta blockers, 3) tachypnea (>20 breaths/min) or need for mechanical ventilation, and 4) leukocytosis (total leukocyte count>11,000/μL) (Anonymous. Critical Care Medicine (1992) 20(6):864-74). Subjects were included in this cohort on the calendar day on which the SIRS criteria were met.

A subject's baseline demographics that were recorded included age, gender, whether medical or surgical diagnosis for admission (according to APACHE m diagnostic codes (KNAUS W A et al. Chest (1991) 100(6):1619-36)), and admission APACHE II score. The following additional data were recorded for each 24 hour period (8 am to 8 am) for 28 days to evaluate organ dysfunction, SIRS, sepsis, and septic shock.

Clinically significant organ dysfunction for each organ system was defined as present during a 24 hour period if there was evidence of at least moderate organ dysfunction using the Brussels criteria (TABLE 2A) (RUSSELL J A et al. Critical Care Medicine (2000) 28(10):3405-11). Because data were not always available during each 24 hour period for each organ dysfunction variable, we used the “carry forward” assumption as defined previously (Anonymous. New England Journal of Medicine (2000) 342(18):1301-8). Briefly, for any 24 hour period in which there was no measurement of a variable, we carried forward the “present” or “absent” criteria from the previous 24 hour period. If any variable was never measured, it was assumed to be normal.

To further evaluate cardiovascular, respiratory, and renal function we also recorded, during each 24-hour period, vasopressor support, mechanical ventilation, and renal support, respectively. Vasopressor use was defined as dopamine>5 μg/kg/min or any dose of norepinephrine, epinephrine, vasopressin, or phenylephrine. Mechanical ventilation was defined as need for intubation and positive airway pressure (i.e. T-piece and mask ventilation were not considered ventilation). Renal support was defined as hemodialysis, peritoneal dialysis, or any continuous renal support mode (e.g. continuous veno-venous hemodialysis). In addition, severity of respiratory dysfunction was assessed, by measuring the occurrence of acute lung injury at the time of meeting the inclusion criteria. Acute lung injury was defined as having a PaO₂/FiO₂ ratio<300, diffuse infiltrates pattern on chest radiograph, and a CVP<18 mm Hg.

To assess duration of organ dysfunction and to correct organ dysfunction scoring for deaths in the 28-day observation period, calculations were made of days alive and free of organ dysfunction (DAF) as previously reported (BERNARD G R et al. New England Journal of Medicine (1997) 336(13):912-8). Briefly, during each 24-hour period for each variable, DAF was scored as 1 if the subject was alive and free of organ dysfunction (normal or mild organ dysfunction, Table 2A). DAF was scored as 0 if the subject had organ dysfunction (moderate, severe, or extreme) or was not alive during that 24-hour period. Each of the 28 days after ICU admission was scored in each subject in this fashion. Thus, the lowest score possible for each variable was zero and the highest score possible was 28. A low score is indicative of more organ dysfunction as there would be fewer days alive and free of organ dysfunction.

Similarly, days alive and free of SIRS (DAF SIRS) were calculated. Each of the four SIRS criteria were recorded as present or absent during each 24 hour period. Presence of SIRS during each 24 hour period was defined by having at least 2 of the 4 SIRS criteria. Sepsis was defined as present during a 24 hour period by having at least two of four SIRS criteria and having a known or suspected infection during the 24 hour period (Anonymous. Critical Care Medicine (1992) 20(6):864-74). Cultures that were judged to be positive due to contamination or colonization were excluded. Septic shock was defined as presence of sepsis plus presence of hypotension (systolic blood pressure<90 mmHg or need for vasopressor agents) during the same 24 hour period.

### Microbiology

Microbiological cultures were taken for any patients who were suspected of having an infection. As this is a cohort of critically ill patients with SIRS, most patients had cultures taken. Positive cultures that were suspected of having been contaminated or colonized were excluded. Positive cultures that were deemed to clinically be clinically irrelevant were also excluded. Cultures were categorized as gram positive, gram negative, fungal or other. The sources of the cultures were respiratory, gastrointestinal, skin, soft tissues or wounds, genitourinary, or endovascular.

Haplotypes and Selection of htSNPs

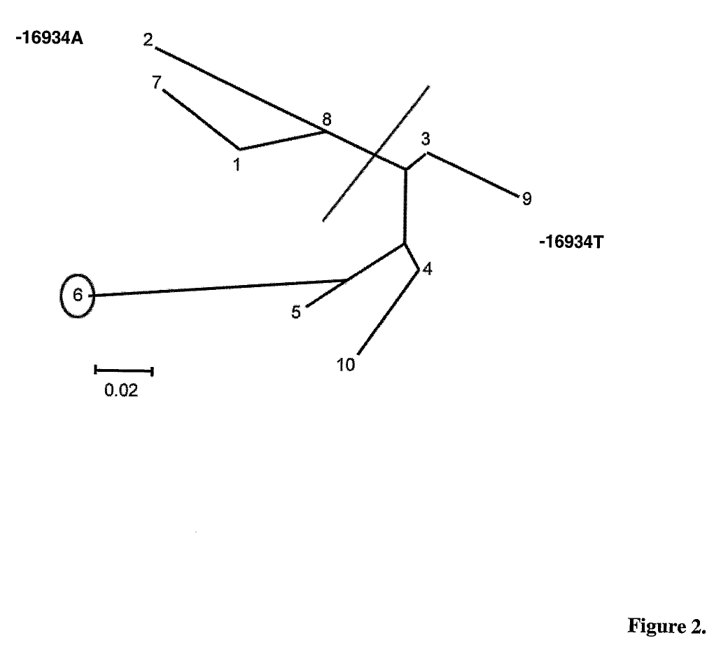

Using unphased Caucasian genotypic data from www.innateimmunity.net/IIPGA/IIPGASNPs (Riva A. and Kohane I S. A Web-Based Tool to Retrieve Human Genome Polymorphisms from Public Databases AMIA 2001 Annual conference, Washington D.C., November 2001), haplotypes were inferred using PHASE software (STEPHENS M. et al. Am J Hum Genet (2001) 68:978-89—see FIG. 1). MEGA 2 (KUMAR S. et al. (2001) 17:1244-5) was then used to infer a phylogenetic tree to identify major haplotype clades for TLR-2 (FIG. 2). Haplotypes were sorted according to the phylogenetic tree and haplotype structure was inspected to choose haplotype tag SNPs (htSNPs) (JOHNSON G C. et al. Nat Genet (2001) 29:233-7; and GABRIEL S B. et al. Science (2002) 296:2225-9). htSNPs were chosen that identified major haplotype clades of TLR-2 in Caucasians were chosen. An htSNP was chosen that identified the 2 major haplotype clades of TLR2 in Caucasians (rs4696480, position -16934 relative to the transcription start site of the TLR2 gene). This SNP was then genotyped in our patient cohort to define major clades. “Tag” SNPs (tSNPs) or “haplotype tag” SNPs (htSNPs) can be selected to uniquely define a clade and serve as markers for all SNPs within haplotypes of the clade.

### Blood Collection/Processing Genotyping

The buffy coat was extracted from whole blood and samples transferred into 1.5 ml cryotubes and stored at −80° C. DNA was extracted from the buffy coat of peripheral blood samples using a QIAamp DNA Blood Maxi Kit (Qiagen™). The genotypic analysis was performed in a blinded fashion, without clinical information. Polymorphisms were genotyped using a real time polymerase chain reaction (PCR) using specific fluorescence-labeled hybridization probes in the ABI Prism 7900 HT Sequence Detection System (Applied Biosystems, Inc.—Livak K J. (1999) Genet Anal 14:143-9). Briefly, the ABI Prism 7900HT uses a 5′ Nuclease Assay in which an allele-specific probe labeled with a fluorogenic reporter dye and a fluorogenic quencher is included in the PCR reaction. The probe is cleaved by the 5′ nuclease activity of Taq DNA polymerase if the probe target is being amplified, freeing the reporter dye and causing an increase in specific fluorescence intensity. Mismatched probes are not cleaved efficiently and thus do not contribute appreciably to the final fluorescent signal. An increase in a specific dye fluorescence indicates homozygosity for the dye-specific allele. An increase in both signals indicated heterozygosity. DNA from lymphocyte cell lines obtained from the Coriell Cell Repository was used to ensure the accuracy of the genotyping. The genotype of these cell lines at G5110A, A5218C and A6235 was determined using the ABI Prism 7900HT Sequence Detection system and compared to the genotype of the same cell lines determined by direct sequencing, given at www.innateimmunity.net/IIPGA/IIPGASNPs (Riva A. and Kohane I S. A Web-Based Tool to Retrieve Human Genome Polymorphisms from Public Databases AMIA 2001 Annual conference, Washington D.C., November 2001).

### Data Collection

Data was recorded for 28 days or until hospital discharge. Raw clinical and laboratory variables were recorded using the worst or most abnormal variable for each 24 hour period with the exception of Glasgow Coma Score, where the best possible score for each 24 hour period was recorded. Missing data on the date of admission was assigned a normal value and missing data after the day one was substituted by carrying forward the previous day's value. Demographic and microbiologic data were recorded. When data collection for each subject was complete, all subject identifiers were removed from all records and the subject file was assigned a unique random number that was cross referenced with the blood samples. The completed raw data file was converted to calculated descriptive and severity of illness scores using standard definitions (i.e. APACHE II and Days alive and free of organ dysfunction calculated using the Brussels criteria). A chi-squared test was used to test for an association between 28-day mortality and haplotype clades. Baseline descriptive characteristics were compared using chi-squared test and ANOVA where appropriate.

### Statistical Analysis

We used a cohort study design. Rates of dichotomous outcomes (28-day mortality, sepsis and shock at onset of SIRS) were compared between haplotype clades using a chi-squared test, assuming a dominant model of inheritance. Differences in continuous outcome variables between haplotype clades were tested using ANOVA. 28-day mortality was further compared between haplotype clades while adjusting for other confounders (age, sex, and medical vs. surgical diagnosis) using a Cox regression model, together with Kaplan-Meier analysis. Haplotype clade relative risk was calculated. This analysis was performed in the entire cohort, and subsequently in sub-groups of subjects who had sepsis at onset of SIRS, and subjects who had septic shock at onset of SIRS. Genotype distributions were tested for Hardy-Weinberg equilibrium (GUO S W. and THOMPSON E A. (1992) 48:361-72). We report the mean and 95% confidence intervals. Statistical significance was set at p<0.05. The p-values as estimated in examples below were derived from three different statistical methods (ANOVA or Kruskal-Wallis test* or ‡ chi-squared test with Yates' continuity correction). For example, P-values for baseline and three-hour cytokine data were derived from the Kruskal-Wallis test (non-parametric analysis) and the p-values for IL1ra corresponds were obtained using the ANOVA method (parametric analysis) comparing means across all genotypes. The data was analyzed using SPSS 11.5 for Windows™ and SigmaStat 3.0 software (SPSS Inc, Chicago, Ill., 2003).

### 3. Examples

### Example 1

### Toll-Like Receptor 2 (TLR-2) Haplotype Analysis for Gram Positive Infection Susceptibility

252 consecutive critically ill patients admitted to the ICU of St. Paul's Hospital were screened for inclusion. Of these, 223 Caucasian patients were successfully genotyped and make up the cohort of this study.

### Haplotype Clade Deduction

Haplotypes were infered from complete sequencing of TLR2 for 23 Caucasians in the Coriell Cell Repository using PHASE software, and two major haplotype clades were identified using MEGA2 software (FIGS. 1 and 2). These haplotype clades could be resolved by genotyping the “haplotype tag” SNP (htSNP) T-16934A (rs4696480) in our cohort of critically ill patients. The minor T allele of this polymorphism marks a clade which encompasses 47% of the haplotypes found in the TLR2 gene (FIG. 1). The genotype frequencies of this polymorphism were similar to frequencies deduced from other available Caucasian data from www.innateimmunity.net/IIPGA/IIPGASNPs (SNP information was retrieved from the Innate Immunity PGA, NHLBI Program in Genomic Applications. Riva A. and Kohane I S. A Web-Based Tool to Retrieve Human Genome Polymorphisms from Public Databases AMIA 2001 Annual conference, Washington D.C., November 2001), and were in Hardy-Weinberg equilibrium (Table 3).

TABLE 3
Genotype Frequencies and Allele Frequencies for TLR-2 htSNP
T-16934A in a Cohort of 223 Critically Ill Adults who had SIRS

| | |
|---|---|
| Genotype | Allele |
| Frequencies | Frequencies |

| | AA | TA | TT | A | T | p° |
|---|---|---|---|---|---|---|
| T-16934A | 25% | 51% | 24% | 51% | 49% | 0.796 |

°exact test of Guo and Thompson to test for Hardy-Weinberg equilibrium

A haplotype clade-based approach was chosen to test the association of polymorphisms to outcome to reduce the potential amount of genotyping almost 30-fold (28 to 1 genotyped SNPs) with little loss of information, as there is less difference between haplotypes within a clade than between haplotypes from different clades (Kareco T R J. and Walley K R. (2003) American Journal of Respiratory and Critical Care Medicine 167:A427). The T-16934A SNP is at a major branch point of the TLR2 haplotype phylogenetic tree and thus splits the haplotypes of TLR2 into 2 clades of approximately equal size (FIG. 2). Haplotype 6 falls into the clade defined by the -16934A allele although it does not appear to be closely related to other haplotypes within the clade defined by the -16934A allele. This may be the result of a reverse mutation in this haplotye from the -16934T allele back to the -16934A allele at some point in history.

### Haplotype Patient Outcome

For the 223 successfully genotyped individuals of the cohort of Caucasian patients who had at least 2 of 4 SIRS criteria, no genotype of TLR2T-16934A was significantly associated with a difference in age, gender, medical vs. surgical diagnosis for admission, or severity of illness at time of admission (as estimated by the APACHE II score) (Table 4).

TABLE 4
Baseline characteristics and mortality of 252 critically ill patients
who had SIRS

| | | | Diagnosis for | | |
|---|---|---|---|---|---|
| | Mean | Gender | admission | Mean | 28-day |
| Genotype | Age | (% Male) | (% Surgical) | APACHE II | Mortality |
| AA | 61 | 65% | 39% | 21 | 34% |
| AT | 59 | 68% | 24% | 19 | 35% |
| TT | 58 | 62% | 25% | 19 | 33% |
| p | NS | NS | 0.03 | NS | NS |

In our cohort of critically ill patients the TLR2-16934A/A genotype was associated with an increased rate of sepsis upon admission to the study. 79% of patients who were homozygous for the -16934A allele had sepsis on day one of the observation period, compared to only 60% of patients who were heterozygous or homozygous for -16934T allele (Fisher's exact, p<0.007). Patients with the -16934A/A genotype had a relative risk of 1.3 of having sepsis on day one (95% CI=1.1-1.6).

The TLR-2 T-16934A genotype was found to be predictive of the occurrence of gram positive cultures in our cohort of critically ill patients. 40% of patients with a gram positive culture were homozygous for the A allele at position -16934, while only 22% of patients who did not have a gram positive culture were homozygous for the A allele at position -16934 of the TLR2 gene (Fisher's exact, p<0.02). Patients who were homozygous for the -16934A allele were twice as likely to have a gram positive culture as those patients who were not homozygous for the -16934A allele (RR=2.0, 95% CI=1.1-3.4). There was furthermore, a specific association of the A/A and A/T genotypes with positive blood cultures. 90% of patients with positive endovascular cultures had the A/A or A/T genotypes at position -16934, while only 74% of patients who had a positive culture from another source (respiratory, gastrointestinal, skin or soft tissues, or genitourinary) had the A/A or A/T genotype (Fisher's exact, p<0.05).

### Example 2

### Toll-Like Receptor 2 (TLR-2) Haplotype Analysis for Ability to Recover from an Inflammatory Condition

TLR-2 T-16934A were examined in a cohort of 638 ICU patients all of whom had SIRS. 25% of patients were AA homozygotes, 48% of patients were AT heterozygotes, and 27% of patients were TT homozygotes. The frequency of the A allele was 49% and the frequency of the T allele was 51% and these alleles were in Hardy Weinberg equilibrium in this population (TABLE 5). There were no differences in age, gender, APACHE II score, and distribution of medical vs. surgical admission status between TLR-2-16934 AA, AT AND TT (TABLE 5).

TABLE 5
Baseline characteristics of critically ill patients having SIRS who were
genotyped for TLR-2 - 16934 T/A.

| | | TLR-2 - | TLR-2 - 16934 |
|---|---|---|---|
| Variable | TLR-2 - 16934 TT | 16934 AT | AA |
| N (%) | 176 (27%) | 305 (48%) | 157 (25%) |
| Age (mean ± | 59 ± 1 | 57 ± 1 | 58 ± 1 |
| SE) | | | |
| Gender (% | 66 | 63 | 64 |
| Male) | | | |
| APACHE II | 24 ± 1 | 23 ± 1 | 23 ± 1 |
| Score (mean ± | | | |
| SE) | | | |
| % Surgical | 24 | 23 | 28 |
| Diagnosis | | | |

There were no differences in baseline characteristics (age, gender, % surgical, APACHE II score) according to genotype.

There was a statistically significant progressive decrease of survival of patients who were TLR-2-16934 AA, vs. TLR-2-16934 AT vs. TLR-2-16934 TT (P value=0.0359).

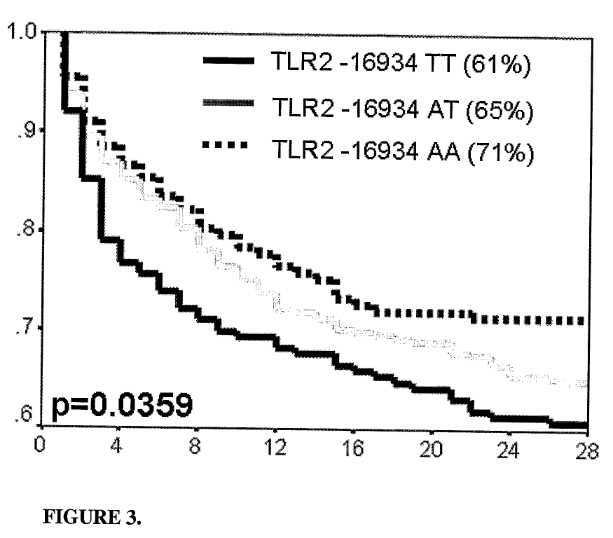

Patients with the AA genotype had a survival of 71%, those with the AT genotype had a 65% survival, and those with a TT genotype had a 60% survival rate at 28 days (TABLE 6, FIG. 3). Thus patients with SIRS having a TLR2 risk genotype (i.e. someone carrying one or more copies of the risk haplotype carrying TLR2-16934T) have a decrease in survival.

TABLE 6
Organ dysfunction of critically ill patients who had SIRS genotyped
for TLR-2 - 16934 T/A.

| | TLR-2 - | TLR-2 - | TLR-2 - 16934 | p |
|---|---|---|---|---|
| Variable | 16934 TT | 16934 AT | AA | Value* |
| 28-Day Survival | 61 | 65 | 71 | 0.036 |
| (%) | | | | |
| Sepsis on | 76 | 81 | 85 | 0.025 |
| Admission (%) | | | | |
| DAF CVS | 15 ± 1 | 16 ± 1 | 17 ± 1 | 0.019 |
| (mean ± SE) | | | | |
| DAF | 17 ± 1 | 18 ± 1 | 19 ± 1 | 0.041 |
| Vasopressors | | | | |
| (mean ± SE) | | | | |
| DAF Inotropes | 19 ± 1 | 20 ± 1 | 21 ± 1 | 0.074 |
| (mean ± SE) | | | | |
| DAF ¾ SIRS | 13 ± 1 | 14 ± 1 | 15 ± 1 | 0.095 |
| (mean ± SE) | | | | |
| DAF Steroids | 15 ± 1 | 16 ± 1 | 17 ± 1 | 0.064 |
| (mean ± SE) | | | | |
| DAF Coagulation | 18 ± 1 | 19 ± 1 | 20 ± 1 | 0.048 |
| (mean ± SE) | | | | |
| DAF INR >1.5 | 17 ± 1 | 18 ± 1 | 20 ± 1 | 0.072 |
| (mean ± SE) | | | | |
| DAF Renal | 17 ± 1 | 18 ± 1 | 19 ± 1 | 0.082 |
| Support | | | | |
| (mean ± SE) | | | | |
| DAF Hepatic | 17 ± 1 | 19 ± 1 | 20 ± 1 | 0.035 |
| (mean ± SE) | | | | |

*All p-values generated using the Kruskal-Wallis test.

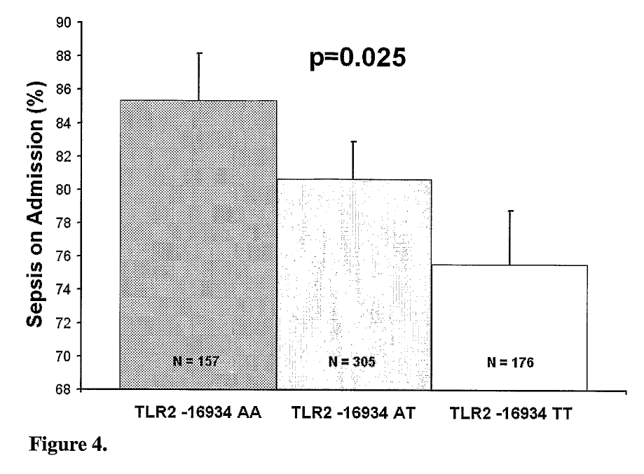

There was a statistically significant (p=0.025) progressive increase in prevalence of sepsis on admission to the ICU in patients who had TLR-2-16934 genotypes TT (76%), AT (81%) and AA (85%) (TABLE 6, FIG. 4).

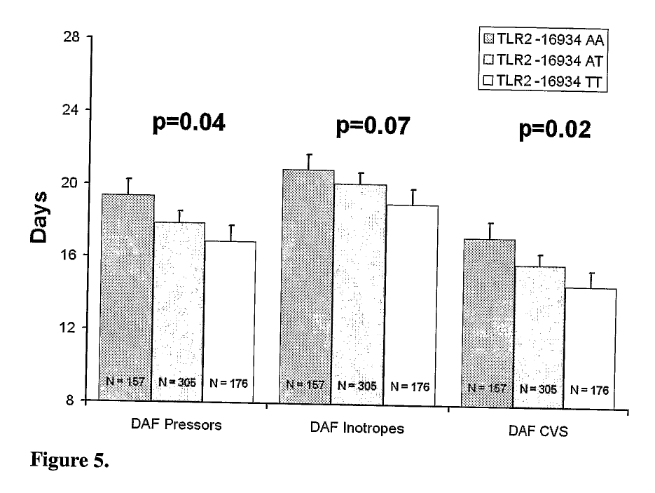

There was a highly significant association of TLR-2-16934 T/A genotype with days alive and free of cardiovascular dysfunction (p=0.019), days alive and free of vasopressors (pressors) (p=0.041) and days alive and free of inotropic agents (inotropes) (p=074) (TABLE 6, FIG. 5). Patients who carried the TLR-2-16934 T allele (AT or TT) had more cardiovascular dysfunction shown as fewer days alive and free of cardiovascular dysfunction, vasopressor use and inotropic agent use. Thus patients with SIRS having a TLR2 risk genotype (i.e. someone carrying one or more copies of the risk haplotype carrying TLR2-16934T) have a statistically significant increase in cardiovascular dysfunction, increased requirement for inotropic agents and a strong trend towards requiring vasopressors.

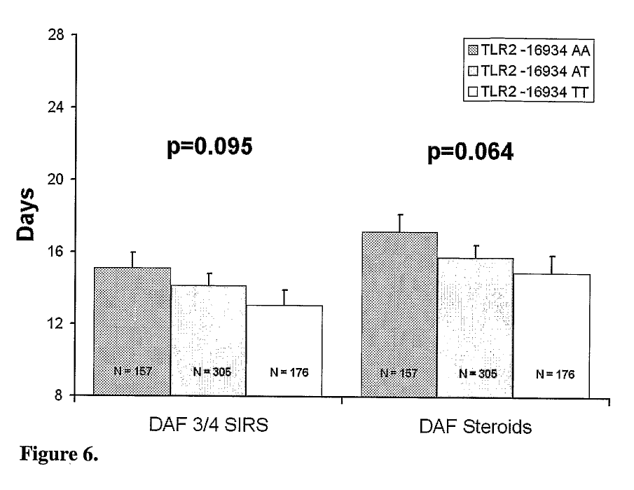

There was a trend toward association between TLR-2-16934 A/T genotype and days alive and free of 3 out of 4 SIRS criteria (p=0.095) and days alive and free of steroid support (p=0.064) (TABLE 6, FIG. 6). Patients who carried the TLR-2-16934 T allele (AT or TT) had more systemic inflammation shown as fewer days alive and free of 3 of 4 SIRS criteria and fewer days alive and free of steroid use.

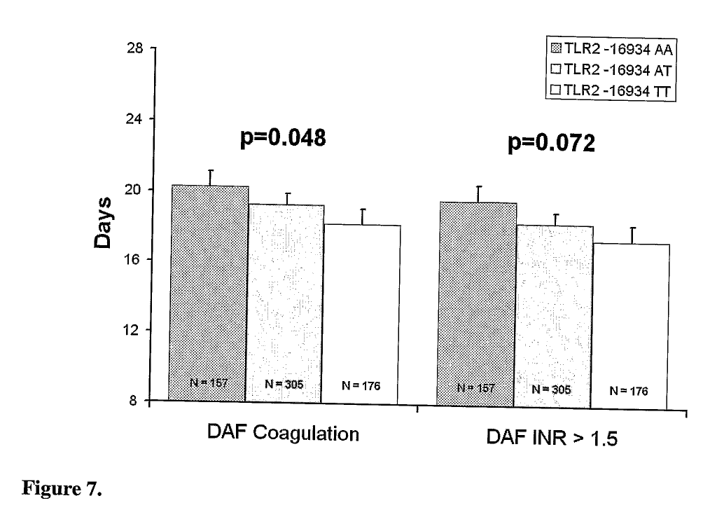

There was a statistically significant association between TLR-2-16935 A/T genotype and days alive and free of coagulation (p=0.048) and a trend toward and association between TLR-2-16945 A/T genotype and days alive and free of INR>1.5 (p=0.072) (TABLE 6, FIG. 7). Patients who carried the TLR-2-16934 T allele (AT or TT) had more coagulation dysfunction shown as fewer days alive and free of coagulation dysfunction and fewer days alive and free of INR>1.5.

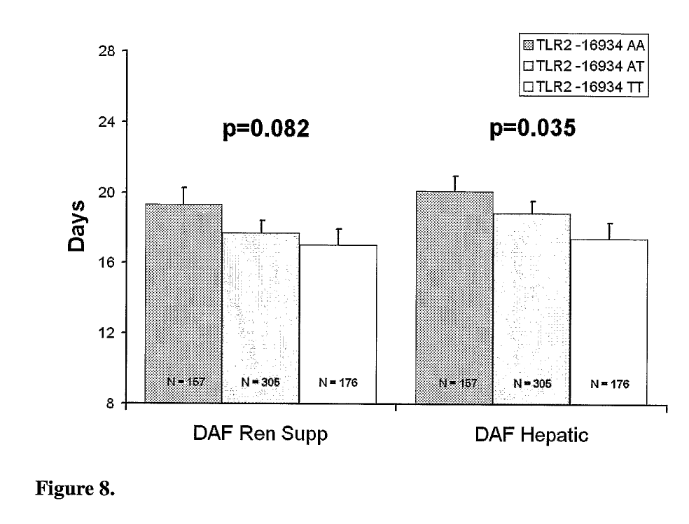

There was a trend toward and association between TLR-2-16934 A/T genotype and days alive and free of renal support (p=0.082) and a significant association between TLR-2-16934 A/T genotype and days alive and free of hepatic dysfunction (p=0.035) (TABLE 6, FIG. 8). Patients who carried the TLR-2-16934 T allele (AT or TT) had more renal dysfunction shown as fewer days alive and free of renal support and more hepatic dysfunction shown as fewer days alive and free of hepatic dysfunction.

### Sepsis Subgroup

We examined the TLR-2 T-16934A polymorphism in a subgroup of 513 critically ill Caucasians all of whom had sepsis as defined in the methods. 26% of patients were AA homozygotes, 48% were AT heterozygotes and 26% were TT homozygotes. The frequency of the A allele was 50% and the frequency of the T allele was also 50%, and these alleles were in Hardy-Weinberg equilibrium (TABLE 7). There were no significant differences in age, gender, APACHE II score or distribution of medical vs. surgical admission status between TLR-2-16934 AA, AT or TT genotype groups (TABLE 7).

TABLE 7
Baseline characteristics of critically ill patients who had sepsis who were
genotyped for TLR-2 - 16934 T/A.

| | TLR-2 - | TLR-2 - | |
|---|---|---|---|
| Variable | 16934 TT | 16934 AT | TLR-2 - 16934 AA |
| N (%) | 133 (26%) | 246 (48%) | 134 (26%) |
| Age (mean ± SE) | 59 ± 1 | 57 ± 1 | 58 ± 1 |
| Gender (% Male) | 67 | 67 | 64 |
| APACHE II Score | 25 ± 1 | 24 ± 1 | 24 ± 1 |
| (mean ± SE) | | | |
| % Surgical | 26 | 24 | 29 |
| Diagnosis | | | |

There were no differences in baseline characteristics (age, gender, % surgical, APACHE II score) according to genotype.

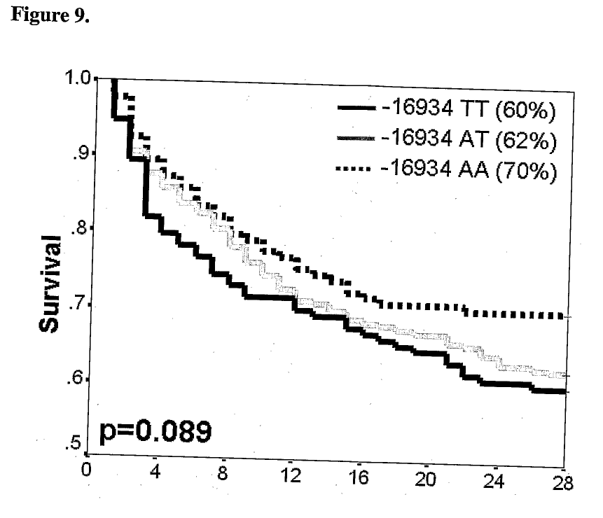

There was a trend toward a progressive decrease in 28-day survival among TLR-2-16934 AA, AT and TT genotype groups (p=0.089). Patients with the AA genotype had a survival of 70%, those with the AT genotype had a survival of 62% and those with the TT genotype had a survival of 60% (TABLE 8, FIG. 9).

TABLE 8
Organ dysfunction of critically ill patients who had sepsis who were
genotyped for TLR-2 - 16934 T/A.

| | TLR-2 - | TLR-2 - | TLR-2 - | |
|---|---|---|---|---|
| Variable | 16934 TT | 16934 AT | 16934 AA | p Value |
| 28-Day | 60 | 62 | 70 | 0.088 |
| Survival (%) | | | | |
| DAF CVS | 14 ± 1 | 15 ± 1 | 17 ± 1 | 0.041 |
| (mean ± SE) | | | | |
| DAF | 17 ± 1 | 17 ± 1 | 19 ± 1 | 0.049 |
| Vasopressors | | | | |
| (mean ± SE) | | | | |
| DAF ¾ SIRS | 12 ± 1 | 13 ± 1 | 14 ± 1 | 0.082 |
| (mean ± SE) | | | | |
| DAF Steroids | 15 ± 1 | 15 ± 1 | 17 ± 1 | 0.092 |
| (mean ± SE) | | | | |
| DAF | 18 ± 1 | 19 ± 1 | 20 ± 1 | 0.084 |
| Coagulation | | | | |
| (mean ± SE) | | | | |
| DAF INR >1.5 | 17 ± 1 | 18 ± 1 | 19 ± 1 | 0.060 |
| (mean ± SE) | | | | |
| DAF Hepatic | 17 ± 1 | 19 ± 1 | 20 ± 1 | 0.066 |
| (mean ± SE) | | | | |
| DAF | 12 ± 1 | 13 ± 1 | 14 ± 1 | 0.071 |
| Respiratory | | | | |
| (mean ± SE) | | | | |
| DAF | 11 ± 1 | 12 ± 1 | 13 ± 1 | 0.099 |
| Mechanical | | | | |
| (mean ± SE) | | | | |

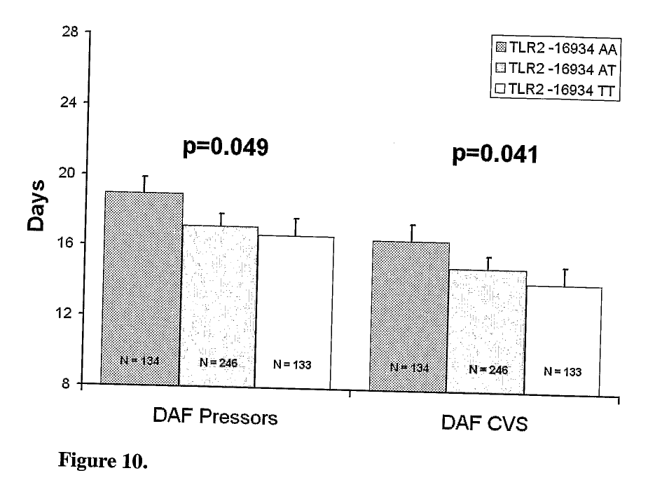

There was a statistically significant association between TLR-2-16934 T/A genotype and days alive and free of vasopressor use (p=0.049) and cardiovascular dysfunction (p=0.041) (TABLE 8, FIG. 10). Patients who had sepsis who carried the TLR-2-16934 T allele (AT or TT) had more cardiovascular dysfunction shown as fewer days alive and free of cardiovascular dysfunction, vasopressor use and inotropic agent use.

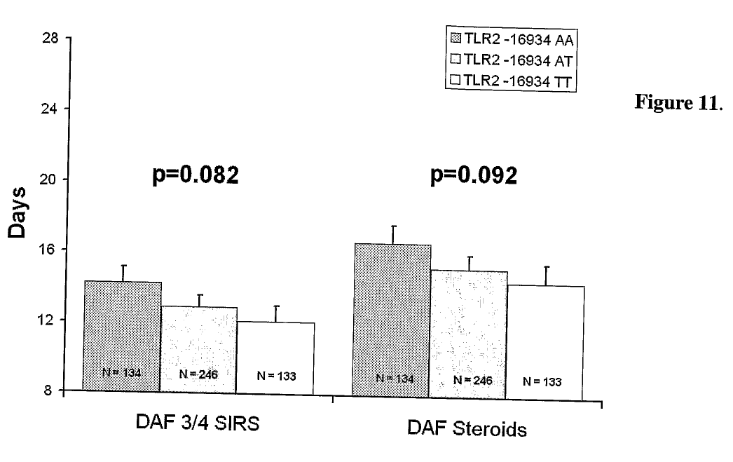

There was a trend toward an association between TLR-2-16934 T/A genotype and days alive and free of 3 out of 4 SIRS criteria (p=0.082) and days alive and free of steroid support (p=0.092) (TABLE 8, FIG. 11). Patients who had sepsis who carried the TLR2-16934 T allele (AT or TT) had more systemic inflammation shown as fewer days alive and free of 3 of 4 SIRS criteria and fewer days alive and free of steroid use.

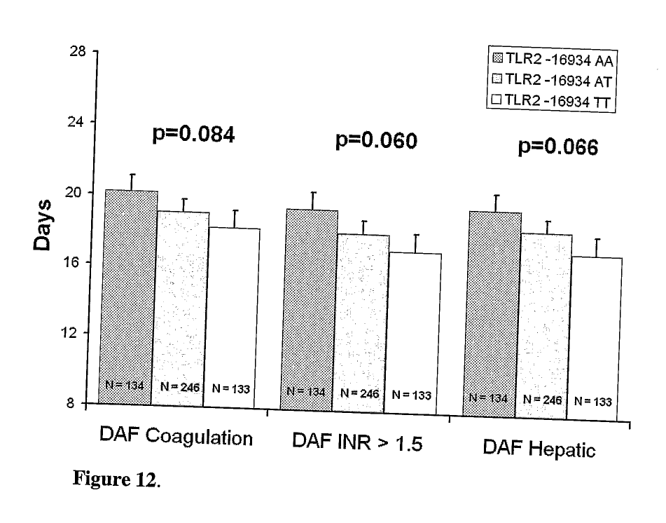

There was a trend toward a significant association between TLR-2-16934 T/A genotype and days alive and free of coagulation (p=0.084), days alive and free of INR>1.5 (p=0.060) and days alive and free of hepatic dysfunction (p=0.066) (TABLE 8, FIG. 12). Patients who had sepsis who carried the TLR-2-16934 T allele (AT or TT) had more coagulation dysfunction shown as fewer days alive and free of coagulation dysfunction and fewer days alive and free of INR>1.5.

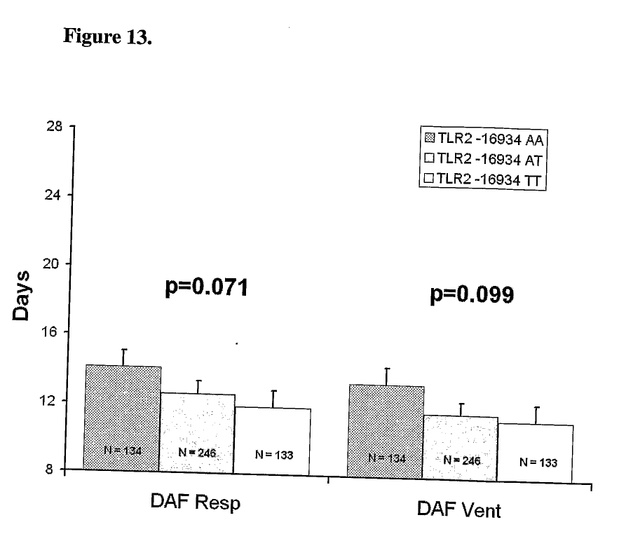

There was a trend toward a significant association between TLR-2-16934 T/A genotype and days alive and free of respiratory dysfunction (p=0.071) and between TLR-2-16934 T/A genotype and days alive and free of mechanical ventilation (p=0.099) (TABLE 8, FIG. 13). Patients who carried the TLR-2-16934 T allele (AT or TT) had more respiratory dysfunction shown as fewer days alive and free of respiratory dysfunction and fewer days alive and free of mechanical ventilation.

### Example 3

### Toll-Like Receptor 2 (TLR-2) Haplotype Analysis for Biological Plausibility Cohort

In this example, the T-16934A allele was studied in non-septic systemic inflammatory response syndrome, in patients following cardiopulmonary bypass surgery (Biological Plausibility Cohort). An evaluation was made whether the risk haplotype -16934T was associated with altered levels of serum IL-8 (a pro-inflammatory cytokine) or levels of serum IL-10 or interleukin 1 receptor antagonist (IL-1ra) levels (IL-10 and IL-1ra being anti-inflammatory cytokines).

### Inclusion Criteria of Biological Plausibility Cohort

Caucasian patients scheduled for first time elective coronary artery bypass grafting requiring cardiopulmonary bypass were included (n=91). Patients undergoing urgent or emergency cardiopulmonary bypass surgery were not included because these patients may have exhibited an inflammatory response to other triggers such as shock. We did not include patients undergoing valve surgery or repeat cardiac surgery because these patients have different pre-operative pathophysiology and often have longer total surgical and cardiopulmonary bypass times. 88 patients were successfully genotyped for TLR-2 T-16934A using the ABI TaqMan™ protocol (Applied Biosystems, Inc.—Livak K J. (1999) Genet Anal 14:143-9).

### Clinical Phenotype of Biological Plausibility Cohort

After induction of anesthesia and placement of systemic and pulmonary artery catheters that were routinely inserted for clinical purposes at our institution, blood was obtained prior to cardiopulmonary bypass for baseline measurement of serum IL-8, IL-10 and IL-1ra using LUMINEX (Oliver K G. et al. (1998) Clinical Chemistry 44(9):2057-60).

Baseline characteristics in the biological plausibility cohort were age, gender, smoking status, presence of diabetes, presence of hypertension, pre-operative ejection fraction, duration of cardiopulmonary bypass and duration of cross-clamp and aprotinin use.

The primary outcome variable for the biological plausibility cohort was change in post-operative IL-8, IL-10 and interleukin 1 receptor antagonist (IL-1ra) concentrations (from 0 hours pre-operatively to 3 hours after surgery). Differences in data were tested using a Kruskal Wallis test (Myles Hollander & Douglas A. Wolfe (1973). Nonparametric statistical inference. New York: John Wiley & Sons. Pages 115-120). Differences were considered significant for p<0.05. The primary distributions of these measurements were skewed at high concentrations. Therefore we log transformed these data. Differences in log-transformed data were tested using ANOVA. All statistical tests were performed using the SPSS statistics software package, version 11.5 (SPSS, Chicago, Ill.).

### Study Rationale

To confirm the clinical observations and to test for evidence of biological plausibility of the observation that TLR-2-16934 T allele (which is associated with worse systemic inflammatory response syndrome, lower survival and greater organ dysfunction in critically ill patients having SIRS and sepsis), we turned to an independent population. Ninety Caucasian patients were studied just prior to and following cardiopulmonary bypass surgery. Cardiopulmonary bypass is associated with an inflammatory response that fulfills the definition of systemic inflammatory response syndrome and is correlated with increased inflammatory cytokine expression. In this population of 88 Caucasians, it was found that the AA, AT and TT genotypes frequencies were 17, 57 and 14 respectively. This population was also in Hardy-Weinberg equilibrium. There were no significant differences between patients by genotype in age, gender distribution, smokers, diabetes, presence of hypertension, preoperative ejection fraction, bypass time, cross-clamp time, and Aprotinin use (TABLE 9).

TABLE 9
Characteristics by genotype of TLR-2-16934 T allele (TT) vs.
TLR-2 - 16934 AA/AT of biological plausibility cohort: in coronary
artery bypass grafting and cardiopulmonary bypass patients.

| Characteristic | TLR-2 - | TLR-2 - | TLR-2 - | |
|---|---|---|---|---|
| Mean ± standard | 16934 | 16934 | 16934 | |
| error of the | AA | AT | TT | |
| mean | N = 17 | N = 57 | N = 14 | P Value |
| Age | 60 ± 1.5 | 66 ± 1 | 65 ± 3 | 0.056* |
| Sex (% Male) | 82 | 68 | 57 | 0.308‡ |
| Smokers (%) | 20 | 17 | 33 | 0.460‡ |
| Diabetes (%) | 40 | 17 | 33 | 0.139‡ |
| Hypertension | 73 | 58 | 50 | 0.425‡ |
| (%) | | | | |
| Pre-op ejection | 0.51 ± 0.03 | 0.51 ± 0.02 | 0.52 ± 0.04 | 0.953* |
| fraction | | | | |
| Bypass time | 1.6 ± 0.1 | 1.8 ± 0.1 | 1.6 ± 0.1 | 0.46* |
| (Hours) | | | | |
| X clamp time | 1.2 ± 0.1 | 1.4 ± 0.1 | 1.2 ± 0.1 | 0.657* |
| (Hours) | | | | |
| Aprotinin use | 0.07 | 0.04 | 0.2 | 0.258‡ |
| (%) | | | | |

*P-values for Age, Ejection Fraction, Bypass Time, and Clamp Time were generated using the Kruskal-Wallis test and
‡P-values for Sex, Smokers, Diabetes, Hypertension, Aprotinin were generated using a chi-squared test with Yates' continuity correction.

Relationships between TLR22T-16934A and serum IL-8, IL-10 and IL-1ra concentrations were then assessed after cardiopulmonary bypass.

### TLR-2 T -16934A and IL-8 Response to Cardiovascular Surgery

There was a significantly greater increase in serum IL-8 of patients according to genotype of TLR-2-16934 (AA. AT, TT IL-8: 41±8 pg/ml, 89±18 pg/ml, and 102±61 pg/ml respectively, p=0.048 linear model for differences in log serum IL-8 concentrations from 0 to 3 hours post-operative comparing TLR-2-16934 AA to TLR-2-1634 AT, see TABLE 10).

TABLE 10
Serum IL-8 levels (mean ± standard error of the mean) by genotype
of TLR-2 T-16934A (i.e. AA, AT and TT) of biological plausibility
cohort: patients who had cardiovascular surgery and cardiopulmonary
bypass.
There was a significantly greater increase in serum IL-8 of patients
according to genotype of - 16934 (AA. AT, TT IL-8: 41 ± 8 pg/ml,
89 ± 18 pg/ml, and 102 ± 61 pg/ml respectively, p = 0.048 linear
model for differences in log serum IL-8 concentrations from 0 to 3 hours
post-operative comparing TLR-2 - 16934 AA to TLR-2 - 1634 AT).

| | IL-8 (pg/mL, Mean ± SE) | | | |
|---|---|---|---|---|
| | TLR 2 - | TLR 2 - | TLR 2 - | |
| | 16934 | 16934 | 16934 | |
| | AA | AT | TT | |
| Time Point | N = 17 | N = 57 | N = 14 | P value |
| 0 hours | 8 ± 3 | 14 ± 2 | 14 ± 5 | 0.355* |
| 3 hours | 49 ± 9 | 103 ± 19 | 116 ± 64 | 0.065* |
| IL-8 | 41 ± 8 | 89 ± 18 (1) | 102 ± 61 | (1) 0.048 |
| Difference | | | | |
| (3 hours − 0 | | | | |
| hours) | | | | |

*P-values for for 0- and 3-hour cytokine levels were generated using the Kruskal-Wallis test.

The TLR-2-16934 T allele was associated with increased pro-inflammatory cytokine (IL-8) response post cardiopulmonary bypass. These findings support and provide a biological plausibility for our findings in the critically ill SIRS patients of associations between TLR-2-16934 T and decreased survival and increased organ dysfunction because of the significantly higher levels of IL-8 associated with the TLR-2-16934 T allele. These results also support our findings of increased risk of infection, especially Gram-positive infection in patients who were TLR-2-16934 A because these patients had lower levels of serum IL-8 after cardiac surgery and IL-8 is an important cytokine in the response to infection.

### TLR-2 T -16934A and IL-10 Response to Cardiovascular Surgery

Relationships between TLR-2 T -16934A and serum IL-10 concentrations were assessed before and after cardiopulmonary bypass. There was a statistically significantly greater increase of serum IL-10 concentrations from 0 to 3 hours post cardiopulmonary bypass in patients who carried the TLR-2 T -16934 T allele compared to patients who were TLR-2 T-16934 AA (TLR-2 T -16934AA, TLR-2 T -16934AT, TLR-2 T -16934TT: 4±2, 13±6, 17±10 pg/ml respectively (mean± standard error of the mean, p=0.034, linear model for differences in log serum IL-8 concentrations from 0 to 3 hours post-operative comparing TLR-2-16934 AA to TLR-2-1634 AT, TABLE 11).

TABLE 11
Serum IL-10 levels (mean ± standard error of the mean) by genotype
of TLR-2 T - 16934A (i.e. AA, AT and TT) of biological plausibility
cohort: patients who had cardiovascular surgery and cardiopulmonary
bypass.

| | IL-10 (pg/mL, Mean ± SE) | | | |
|---|---|---|---|---|
| | TLR-2 - | TLR-2 - | TLR-2 - | |
| | 16934 | 16934 | 16934 | |
| | AA | AT | TT | |
| Time Point | N = 17 | N = 57 | N = 14 | P value |
| 0 hours | 1 ± 1 | 2 ± 0.5 | 1 ± 1 | 0.287* |
| 3 hours | 5 ± 2 | 16 ± 6 | 17 ± 10 | 0.658* |
| IL-10 | 4 ± 2 | 13 ± 6 | 17 ± 10 (1) | (1) 0.034 |
| Difference | | | | |
| (3 hours − 0 | | | | |
| hours) | | | | |

*P-values for for 0- and 3-hour cytokine levels were generated using the Kruskal-Wallis test.

Serum IL-10 concentrations shown as mean± standard error of the mean (SE). There was a statistically significantly greater increase of serum IL-10 concentrations from 0 to 3 hours post cardiopulmonary bypass in patients who carried the TLR-2 T -16934 T allele compared to patients who were TLR-2 T -16934AA (TLR-2 T -16934AA, TLR-2 T -16934AT, TLR-2 T -16934TT: 4±2, 13±6, 17±10 pg/ml respectively (mean± standard error of the mean), p=0.034, linear model for differences in log serum IL-8 concentrations from 0 to 3 hours post-operative comparing TLR-2-16934 TT to TLR-2-1634 AA.

TLR-2 T -16934A and Interleukin 1 receptor Antagonist (IL-1ra) Response to Cardiovascular Surgery

There was a statistically significantly greater increase of serum IL-1ra concentrations from 0 to 3 hours post cardiopulmonary bypass in patients who carried the TLR-2 T -16934 A allele compared to patients who were TLR-2 T -16934TT (TLR-2 T -16934AA, TLR-2 T -16934AT, TLR-2 T -16934TT: 28787±5298, 33305±2217, 17354±7289 pg/ml respectively (mean± standard error of the mean, p=0.03, linear model for differences in log serum IL-8 concentrations from 0 to 3 hours post-operative comparing TLR-2-16934 AA, to TLR-2-16934 AT, to TLR-2-1634 TT) (TABLE 12).

TABLE 12
Serum IL-1ra levels (mean ± standard error of the mean) by genotype
of TLR-2 T - 16934A (i.e. AA, AT and TT) of biological plausibility
cohort: in cardiovascular surgery and cardiopulmonary bypass patients.

| | IL-1ra (pg/mL, Mean ± SE) | | | |
|---|---|---|---|---|
| | TLR-2 - | TLR-2 - | TLR-2 - | |
| | 16934 | 16934 | 16934 | |
| | AA | AT | TT | P |
| Time Point | N = 17 | N = 57 | N = 14 | value |
| 0 hours | 2028 ± 632 | 1528 ± 137 | 4806 ± 3419 | 0.917* |
| 3 hours | 29526 ± 4903 | 34855 ± 2154 | 23699 ± 5253 | 0.115* |
| IL-1ra | 28787 ± 5298 | 33305 ± 2217 | 17354 ± 7289 | 0.03 |
| Difference | | | | |
| (3 hours − 0 | | | | |
| hours) | | | | |

*P-values for for 0- and 3-hour cytokine levels were generated using the Kruskal-Wallis test.

Serum IL-1ra concentrations shown as mean± standard error of the mean (SE). There was a statistically significantly greater increase of serum IL-1ra concentrations from 0 to 3 hours post cardiopulmonary bypass in patients who carried the TLR-2 T -16934 A allele compared to patients who were TLR-2 T -16934TT (TLR-2 T -16934AA, TLR-2 T -16934AT, TLR-2 T -16934TT: 28787±5298, 33305±2217, 17354±7289 pg/ml respectively (mean± standard error of the mean), p=0.03, linear model for differences in log serum IL-8 concentrations from 0 to 3 hours post-operative comparing TLR-2 201 AA, to TLR-2 201 AT, to TLR-2-1634 TT).

The association between TLR-2-16934 T and adverse clinical outcome in the cohort of patients who had SIRS is given further relevance by the important associations between TLR-2-16934 T and serum levels of IL-8 in an analogous but completely independent cohort that had a non-septic systemic inflammatory response syndrome, i.e. patients in whom systemic inflammatory response syndrome was induced by cardiac surgery and cardiopulmonary bypass. We found evidence for association of TLR-2-16934 T allele with greater post-operative pro-inflammatory cytokine response, as reflected by serum IL-8. The increased serum IL-8 levels in patients with the TLR-2-16934 T allele adds biological plausibility, because IL-8 is a key pro-inflammatory cytokine.

The increased cytokine concentrations (IL-8) three hours after cardiopulmonary bypass indicate that there is a rapid increase in synthesis and/or release of cytokines and that this response is amplified in patients who had TLR-2-16934 T allele. The results indicate that patients with the TLR-2-16934 T allele have an increased IL-8 response to an inciting stimulus.

IL-10 is an anti-inflammatory cytokine that increases after surgery and sepsis and is a marker of worse outcome such as decreased survival from sepsis. The finding of greater increase of IL-10 after cardiovascular surgery in patients who carried the TLR-2-16934 T allele (AT/FI) compared to patients who were TLR-2-16934 AA also provides biological plausibility to our findings of decreased survival and greater organ dysfunction of patients who have SIRS who carried the TLR-2-16934 T allele (AT/TT) allele (also called the TLR-2 risk genotype).

Interleukin 1 receptor antagonist (IL-1ra) is an anti-inflammatory cytokine that increases after sepsis. Interleukin 1 receptor antagonist binds to the interleukin 1 receptor and prevents interleukin 1β—a potent pro-inflammatory cytokine—from binding and inducing it's pro-inflammatory actions. Therefore, increased serum levels of IL-1ra diminish the actions of IL-1β. Thus, a diminished response of patients who have the TLR-2-16943 T allele provides further biological plausibility that the patients who carried the TLR-2-16943 T allele had a greater overall inflammatory response to an inciting stimulus (i.e. cardiovascular surgery).

The findings with serum IL-1ra were derived in an independent cohort of patients who had cardiovascular surgery whereas the associations with survival and organ dysfunction were in a separate cohort.

Subjects who present with an inflammatory condition such as SIRS, and who carry a risk genotype, are more likely to benefit from treatment significantly more than persons with non-risk or protective genotypes.

### Clinical Implications

Subjects with sepsis, severe sepsis or SIRS may be genotyped to determine their 201 TLR-2 genotype or the genotype of polymorphism site in linkage disequilibrium with this SNP. Subjects could then be classified by genotype into a risk category regarding their unique risk of death by genotype or their susceptibility of developing a gram positive infection.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims. All patents, patent applications and publications referred to herein are hereby incorporated by reference.

1. A method for making a prognosis in a subject of (i) enhanced recovery from an inflammatory condition, or (ii) increased at risk of developing, the inflammatory condition,
the method comprising determining a genotype defined by one or more polymorphic
sites in the toll-like receptor 2 (TLR-2) nucleic acid sequence in the subject, wherein said genotype is (a) a protective genotype that is predictive or indicative of an enhanced ability of the subject to recover from the inflammatory condition, or (b) a risk genotype that is predictive or indicative of said increased risk for developing the inflammatory condition.

2. The method of claim 1, wherein the one or more polymorphic sites includes position 201 of SEQ ID NO: 1 or a polymorphic site in linkage disequilibrium therewith.

3. The method of claim 1, further comprising comparing the determined genotype with genotypes that are known to be prognostic for recovery from an inflammatory condition.

4. The method of claim 1, further comprising ascertaining the TLR-2 sequence of the subject.

5. The method of claim 1, wherein said genotype determination is performed on a nucleic acid sample from the subject.

6. The method of claim 5, further comprising the step of obtaining the nucleic acid sample from the subject.

7. The method of claim 1, wherein said genotype is determined by one or more of the following methods:
(a) restriction fragment length analysis;
(b) sequencing;
(c) hybridization;
(d) oligonucleotide ligation assay;
(e) ligation rolling circle amplification;
(f) 5′ nuclease assay;
(g) polymerase proofreading methods;
(h) allele specific PCR; and
(i) reading sequence data.

8. The method of claim 1, wherein the risk genotype of the subject is predictive or indicative of (a) decreased likelihood of recovery from the inflammatory condition or (b) increased risk of having a poor outcome from the inflammatory condition.

9. The method of claim 8, wherein the subject is critically ill and the presence of the risk genotype is predictive or indicative of severe cardiovascular or respiratory dysfunction.

10. The method of claim 8, wherein the risk genotype comprises at least one T nucleotide at position 201 of SEQ ID NO: 1.

11. (canceled)

12. The method of claim 1, wherein the subject is critically ill and the protective genotype is predictive or indicative of less severe cardiovascular or respiratory dysfunction.

13. The method of claim 1, wherein the protective genotype is defined as homozygosity for the A nucleotide at position 201 of SEQ ID NO: 1.

14. The method of claim 1, wherein the inflammatory condition is one that is due to, or associated with: Gram-positive, Gram-negative, culture-negative or fungal sepsis; septicemia; septic shock; fever; bacterial viral, fungal or parasitic infection including Group A streptococcus infection; inflammation due to trauma, surgery or a medical or surgical condition associated with increased risk of infection or sepsis; pneumonia; systemic inflammatory response syndrome (SIRS); Acute Respiratory Distress Syndrome (ARDS); acute lung injury; pancreatitis; bacteremia, including meningococcemia; peritonitis; bowel infection; abdominal abscess; surgery; chronic inflammatory disease; ischemia; tissue damage due to (i) disease, (ii) chemotherapy (iii) radiotherapy, or a reaction to an ingested, inhaled, infused, injected, or delivered substance; glomerulonephritis; an opportunistic infection; kidney failure and dialysis; immunosuppressive therapy; immunocompromise; endocarditis; cystic fibrosis; diabetes mellitus; chronic renal failure; bronchiectasis; chronic obstructive pulmonary disease (COPD); chronic bronchitis; emphysema; post-pump syndrome; cardiac stun syndrome; myocardial infarction; stroke; congestive heart failure; hepatitis; cirrhosis; epiglotittis; gas gangrene; toxic shock syndrome; mycobacterial tuberculosis; Pneumocystic carinii pneumonia; Leishmaniasis; hemolytic uremic syndrome; Dengue hemorrhaigic fever; pelvic inflammatory disease; Legionella; Lyme disease; Influenza A; Epstein-Barr virus; encephalitis; autoimmunity and inflammation due to rheumatoid arthritis, osteoarthritis, or systemic lupus erythematosus; inflammatory bowel disease, idiopathic pulmonary fibrosis, sarcoidosis, hypersensitivity pneumonitis, systemic vasculitis, Wegener's granulomatosis; an organ or tissue transplant and/or transplant rejection; graft-versus-host disease; sickle cell anemia; nephrotic syndrome; or toxicity caused by monoclonal antibody or cytokine therapy.

15. The method of claim 14, wherein the inflammatory condition is SIRS.

16. A method of identifying a polymorphism in a TLR-2 sequence that correlates with or is associated with prognosis of recovery from an inflammatory condition in a subject, the method comprising:
(a) obtaining TLR-2 sequence information from a plurality of subjects with an inflammatory condition;
(b) based on the sequence information of (a), identifying at least one site of polymorphism in the TLR-2 sequence;
(c) determining genotypes defined by said at least one polymorphism for individual subjects;
(d) determining recovery ability of individual subjects from the inflammatory condition; and
(e) correlating the genotypes determined in step (c) with the subjects' recovery abilities determined in step (d),
thereby identifying said polymorphisms in said TLR-2 sequence.

17. The method of claim 16, wherein the inflammatory condition is one that is due to, or associated with: Gram-positive, Gram-negative, culture-negative or fungal sepsis; septicemia; septic shock; fever; bacterial viral, fungal or parasitic infection including Group A streptococcus infection; inflammation due to trauma, surgery or a medical or surgical condition associated with increased risk of infection or sepsis; pneumonia; systemic inflammatory response syndrome (SIRS); Acute Respiratory Distress Syndrome (ARDS); acute lung injury; pancreatitis; bacteremia including meningococcemia; peritonitis; bowel infection; abdominal abscess; surgery; chronic inflammatory disease; ischemia; tissue damage due to (i) disease, (ii) chemotherapy (iii) radiotherapy, or a reaction to an ingested, inhaled, infused, injected, or delivered substance; glomerulonephritis; an opportunistic infection; kidney failure and dialysis; immunosuppressive therapy; immunocompromise; endocarditis; cystic fibrosis; diabetes mellitus; chronic renal failure; bronchiectasis; chronic obstructive pulmonary disease (COPD); chronic bronchitis; emphysema; post-pump syndrome; cardiac stun syndrome; myocardial infarction; stroke; congestive heart failure; hepatitis; cirrhosis; epiglotittis; gas gangrene; toxic shock syndrome; mycobacterial tuberculosis; Pneumocystic carinii pneumonia; Leishmaniasis; hemolytic uremic syndrome; Dengue hemorrhaigic fever; pelvic inflammatory disease; Legionella; Lyme disease; Influenza A; Epstein-Barr virus; encephalitis; autoimmunity and inflammation due to rheumatoid arthritis, osteoarthritis, or systemic lupus erythematosus; inflammatory bowel disease, idiopathic pulmonary fibrosis, sarcoidosis, hypersensitivity pneumonitis, systemic vasculitis, Wegener's granulomatosis; an organ or tissue transplant and/or transplant rejection; graft-versus-host disease; sickle cell anemia; nephrotic syndrome; or toxicity caused by monoclonal antibody or cytokine therapy.

18. A kit useful for determining a genotype of a subject or subjects at a defined polymorphic nucleotide position in a TLR-sequence from the subject or subjects, which genotype is associated with a prognosis of the subject's ability to recover from an inflammatory condition, the kit comprising;
(a) a restriction enzyme with specificity that distinguishes alternate nucleotides at the polymorphic site or sites; or
(b) a labeled oligonucleotide having sufficient complementarity to an alternate nucleotide sequence at the polymorphic site such that the oligonucleotide hybridizes in a distinguishable manner to a sequence that comprises said alternate nucleotide sequence, thereby permitting determination of the genotype at the polymorphic site; and
(c) optionally, instructions for use of said enzyme and/or said oligonucleotides in determining the genotype.

19. The kit of claim 18, wherein the polymorphic site is at nucleotide position 201 of SEQ ID NO:1.

20. The kit of claim 18 further comprising an oligonucleotide primer or a set of oligonucleotides suitable to amplify a region flanking the polymorphic site.

21. The kit of claim 20, further comprising a polymerization agent that promotes or permits nucleotide polymerization.

22. A method for identifying subjects as being suitable for a trial that tests efficacy of a candidate drug known to be, or suspected of being, useful for treating an inflammatory disease or condition, the method comprising
(a) determining a genotype defined by one or more polymorphic sites in the TLR-2 sequence for each of said subjects, wherein said genotype is indicative of the subject's recovery ability from the inflammatory disease or condition, and
(b) sorting subjects into a suitable and unsuitable group for said trial based on the subjects' genotype.

23. A method for testing a candidate drug for its efficacy in the treatment of an inflammatory disease or condition wherein said disease or condition is associated with a genotype defined by a polymorphism in a TLR-2 gene, comprising:
(a) identifying subjects that are suitable for a trial that tests said candidate drug in accordance with of claim 22 ; and
(b) administering the candidate drug to each of said subjects, and comparing the subjects' responses to said candidate drug in comparison with the subjects' genotype,
thereby testing said candidate drug.

24. The method of claim 23, wherein a subject's response to the candidate drug is measured as the ability to recover from the inflammatory condition.

25. The method of claim 22 wherein the inflammatory disease or condition is associated with a gram positive infection.

26. The method of claim 23 wherein the inflammatory disease or condition is associated with a gram positive infection.

27. The method of claim 24 method of claim 1 wherein the inflammatory condition is a result of a gram positive infection wherein if said genotype is a risk genotype, it is indicative of the subject's risk of gram positive infection.

28-39. (canceled)

40. The method of claim 1 wherein the inflammatory condition is a result of a gram positive infection, wherein if said genotype is a risk genotype, it is indicative of the subject's risk of gram positive infection.

41. The method of claim 40, wherein the polymorphic site is at position 201 of SEQ ID NO:1.

42. The method of claim 40, further comprising ascertaining the TLR-2 gene sequence of the subject.

43. The method of claim 40, wherein said genotype determination is performed on a nucleic acid sample from the subject.

44. The method of claim 43, further comprising the step of obtaining the nucleic acid sample from the subject.

45. The method of claim 40, wherein said genotype is determined by one or more of the following techniques:
(a) restriction fragment length analysis;
(b) sequencing;
(c) hybridization;
(d) oligonucleotide ligation assay;
(e) ligation rolling circle amplification;
(f) 5′ nuclease assay;
(g) polymerase proofreading methods;
(h) allele specific PCR; and
(i) reading sequence data.

46. The method of claim 40, wherein the risk genotype of the subject is predictive or indicative of the subject's risk of developing a gram positive infection.

47. The method of claim 46, wherein the risk genotype has at least one A nucleotide at position 201 of SEQ ID NO: 1.

48. The method of claim 46, wherein the protective genotype is defined as homozygosity for T at position 201 of SEQ ID NO:1.

49-51. (canceled)

52. An array of nucleic acid molecules immobilized to a solid support, the array comprising:
(a) a first set of oligonucleotides that
(i) hybridize to a nucleic acid molecule consisting of SEQ ID NO:1 in which the nucleotide at position 201 is A, under conditions wherein
(ii) the oligonucleotides of the first set do not substantially hybridize to a nucleic acid molecule consisting of SEQ ID NO:1 in which the nucleotide at position 201 is T; and/or
(b) a second set of oligonucleotides that
(i) hybridize to a nucleic acid molecule consisting of SEQ ID NO:1, in which the nucleotide at position 201 is T, under conditions wherein
(ii) the oligonucleotides of the second set will not substantially hybridize to a nucleic acid molecule consisting of SEQ ID NO: 1 in which the nucleotide at position 201 is A.

53-55. (canceled)
